(19) United States
(12) Patent Application Publication
(10) Pub. No.: US 2013/0236419 A1
(43) Pub. Date: Sep. 12, 2013

(54) COMPOSITIONS AND METHODS RELATED TO ATTENUATED STAPHYLOCOCCAL STRAINS

(71) Applicants: Olaf Schneewind, Chicago, IL (US); Hye-Young Kim, Rancho, CA (US); Dominique M. Missiakas, Chicago, IL (US); Hwan Keun Kim, Naperville, IL (US)

(72) Inventors: Olaf Schneewind, Chicago, IL (US); Hye-Young Kim, Rancho, CA (US); Dominique M. Missiakas, Chicago, IL (US); Hwan Keun Kim, Naperville, IL (US)

(73) Assignee: THE UNIVERSITY OF CHICAGO, Chicago, IL (US)

(21) Appl. No.: 13/821,937

(22) PCT Filed: Sep. 9, 2011
(86) PCT No.: PCT/US2011/051090

§ 371 Date: Jun. 3, 2013

Related U.S. Application Data
(60) Provisional application No. 61/381,363, filed on Sep. 9, 2010.
(60) Provisional application No. 61/435,584, filed on Jan. 24, 2011.

(51) Int. Cl. A61K 39/085

(52) U.S. Cl. CPC A61K 39/085
USPC 424/85.4; 435/252.1; 435/252.3; 424/243.1; 536/23.7

(57) ABSTRACT

The present invention concerns methods and compositions for treating or preventing a bacterial infection, particularly infection by a Staphylococcus bacterium. The invention provides methods and compositions for stimulating an immune response against the bacteria. In certain embodiments, the methods and compositions involve attenuated S. aureus strains having deletions in their genome, such as in the srtA and saeR regions.

This application claims the benefit of priority to U.S. Provisional Patent Application Ser. No. 61/381,363, filed Sep. 9, 2010, and U.S. Provisional Patent Application Ser. No. 61/435,584, filed Jan. 24, 2011, the entire contents of all are hereby incorporated by reference.

This invention was made with government support under AI057153, AI052474, and GM007281 awarded by the National Institutes of Health. The government has certain rights in the invention.

### BACKGROUND OF THE INVENTION

I. Field of the Invention

The present invention relates generally to the fields of immunology, microbiology, and pathology. More particularly, it concerns methods and compositions involving attentuated bacterial variants which can be used to invoke an immune response against the bacteria.

II. Background

The number of both community acquired and hospital acquired infections have increased over recent years with the increased use of intravascular devices. Hospital acquired (nosocomial) infections are a major cause of morbidity and mortality, more particularly in the United States, where it affects more than 2 million patients annually. The most frequent infections are urinary tract infections (33% of the infections), followed by pneumonia (15.5%), surgical site infections (14.8%) and primary bloodstream infections (13%) (Emorl and Gaynes, 1993).

The major nosocomial pathogens include Staphylococcus aureus, coagulase-negative Staphylococci (mostly Staphylococcus epidermidis), enterococcus spp., Escherichia coli and Pseudomonas aeruginosa. Although these pathogens cause approximately the same number of infections, the severity of the disorders they can produce combined with the frequency of antibiotic resistant isolates balance this ranking towards S. aureus and S. epidermidis as being the most significant nosocomial pathogens.

Staphylococci can cause a wide variety of diseases in humans and other animals through either toxin production or invasion. Staphylococcal toxins are also a common cause of food poisoning, as the bacteria can grow in improperly-stored food. Staphylococcus epidermidis is a normal skin commensal which is also an important opportunistic pathogen responsible for infections of impaired medical devices and infections at sites of surgery. Medical devices infected by S. epidermidis include cardiac pacemakers, cerebrospinal fluid shunts, continuous ambulatory peritoneal dialysis catheters, orthopedic devices and prosthetic heart valves.

Staphylococcus aureus is the most common cause of nosocomial infections with a significant morbidity and mortality. It is the cause of some cases of osteomyelitis, endocarditis, septic arthritis, pneumonia, abscesses, and toxic shock syndrome. S. aureus can survive on dry surfaces, increasing the chance of transmission. Any S. aureus infection can cause the staphylococcal scalded skin syndrome, a cutaneous reaction to exotoxin absorbed into the bloodstream. It can also cause a type of septicemia called pyaemia that can be life-threatening. Problematically, Methicillin-resistant Staphylococcus aureus (MRSA) has become a major cause of hospital-acquired infections.

S. aureus and S. epidermidis infections are typically treated with antibiotics, with penicillin being the drug of choice, whereas vancomycin is used for methicillin resistant isolates. The percentage of staphylococcal strains exhibiting wide-spectrum resistance to antibiotics has become increasingly prevalent, posing a threat for effective antimicrobial therapy. In addition, the recent emergence of vancomycin resistant S. aureus strain has aroused fear that MRSA strains are emerging and spreading for which no effective therapy is available.

An alternative to antibiotic treatment for staphylococcal infections is under investigation that uses antibodies directed against staphylococcal antigens. This therapy involves administration of polyclonal antisera (WO00/15238, WO00/12132) or treatment with monoclonal antibodies against lipoteichoic acid (WO98/57994).

An alternative approach would be the use of active vaccination to generate an immune response against staphylococci. The S. aureus genome has been sequenced and many of the coding sequences have been identified (WO02/094868, EP0786519), which can lead to the identification of potential antigens. The same is true for S. epidermidis (WO01/34809). As a refinement of this approach, others have identified proteins that are recognized by hyperimmune sera from patients who have suffered staphylococcal infection (WO01/98499, WO02/059148).

S. aureus secretes a plethora of virulence factors into the extracellular milieu (Archer, 1998; Dinges et al., 2000; Foster, 2005; Shaw et al., 2004; Sibbald et al., 2006). Like most secreted proteins, these virulence factors are translocated by the Sec machinery across the plasma membrane. Proteins secreted by the Sec machinery bear an N-terminal leader peptide that is removed by leader peptidase once the pre-protein is engaged in the Sec translocon (Dalbey and Widmer, 1985; van Wely et al., 2001). Recent genome analysis suggests that Actinobacteria and members of the Firmicutes encode an additional secretion system that recognizes a subset of proteins in a Sec-independent manner (Pallen, 2002). ESAT-6 (early secreted antigen target 6 kDa) and CFP-10 (culture filtrate antigen 10 kDa) of Mycobacterium tuberculosis represent the first substrates of this novel secretion system termed ESX-1 or 5 nm in M. tuberculosis (Andersen et al., 1995; Hsu et al., 2003; Pym et al., 2003; Stanley et al., 2003). In S. aureus, two ESAT-6 like factors designated EsxA and EsxB are secreted by the Ess pathway (ESAT-6 secretion system) (Burts et al., 2005).

The first generation of vaccines targeted against S. aureus or against the exoproteins it produces have met with limited success (Lee, 1996). There remains a need to develop effective vaccines against staphylococcal infections. Thus, additional compositions for treating staphylococcal infections are also needed.

### SUMMARY OF THE INVENTION

Thus, in accordance with the present invention, there is provided an isolated staphylococcal bacterium that does not express a functional SpA, srtA adsA and/or agrA polypeptide. The bacterium may not express SpA and adsA, or may not express SpA and srtA, or may not express agrA and adsA, or may not express SpA and agrA, or may not express srtA and agrA, or may not express adsA and srtA, or may not express SpA, srtA, and adsA, or may not express srtA, adsA and agrA, or may not express SpA, srtA and agrA, or may not express SpA, agrA and adsA, or may not express SpA, srtA, adsA and agrA. The bacterium may lack at least part of a coding region for a SpA, adsA, srtA or agrA polypeptide, may contain a point mutation in a coding region for a SpA, adsA, srtA or agrA polypeptide, such as a point mutation that introduces a stop codon, or may contain an insertion into a coding region for a SpA, adsA, srtA or agrA polypeptide, such as a transposon. The bacterium may further comprise a heterologous drug susceptibility determinant. The bacterium may be S. aureus.

Also provided are method of making a vaccine comprising the step of formulating a bacterium as described above in a pharmaceutically acceptable excipient. Another embodiment comprises the use of a bacterium as described above in the manufacture of a vaccine for treatment or prevention of staphylococcal infection. A further embodiment comprises a pharmaceutical composition comprising a bacterium as described above.

In another embodiment, there is provided a live attenuated vaccine comprising a staphylococcal bacterium that does not express a functional SpA, adsA, srtA and/or agrA polypeptide. The bacterium may not express SpA and adsA, or may not express SpA and srtA, or may not express agrA and adsA, or may not express SpA and agrA, or may not express srtA and agrA, or may not express adsA and srtA, or may not express SpA, srtA, and adsA, or may not express srtA, adsA and agrA, or may not express SpA, srtA and agrA, or may not express SpA, agrA and adsA, or may not express SpA, srtA, adsA and agrA. The bacterium may lack at least part of a coding region for a SpA, adsA, srtA or agrA polypeptide, may contain a point mutation in a coding region for a SpA, adsA, srtA or agrA polypeptide, such as a point mutation that introduces a stop codon, or may contain an insertion into a coding region for a SpA, adsA, srtA or agrA polypeptide, such as a transposon. The bacterium may further comprise a heterologous drug susceptibility determinant. The bacterium may be S. aureus.

Also provided is a method of preventing or treating staphylococcal infection comprising the step of administering a vaccine as described above to a patient in need thereof. Another embodiment comprises the use of a vaccine as described above in the treatment or prevention of staphylococcal infection. Yet another embodiment comprises a kit comprising a vaccine as described above.

A further embodiment involves provides of a nucleic acid encoding the genome of a staphylococcal bacterium that does not express a functional SpA, adsA, srtA and/or agrA polypeptide. The genome may lack at least part of a coding region for a SpA, adsA, srtA or agrA polypeptide, may contain a point mutation in a coding region for a SpA, adsA, srtA or agrA polypeptide, such as where the point mutation introduces a stop codon, or may contain an insertion into a coding region for a SpA, adsA, srtA or agrA polypeptide, such as a transposon. The nucleic acid may further comprise a heterologous drug susceptibility determinant. The staphylococcal bacterium may be S. aureus.

Still an additional embodiment comprises a method for eliciting an immune response against a staphylococcus bacterium in a subject comprising providing to the subject an effective amount of a vaccine as described above. The method may further comprise administering to the subject a biological response modifier, such as an adjuvant, a cytokine or interleukin. The method may further comprise administering the vaccine more than one time to the subject. The vaccine may be administered orally, parenterally, subcutaneously, intramuscularly, or intravenously. The subject may be a mammal (including non-human) or a human. The immune response may be a protective immune response.

In still yet an additional embodiment, there is provided a method for treating a staphylococcal infection in a subject comprising providing to a subject having, suspected of having or at risk of developing a staphylococcal infection an effective amount of a vaccine as described above. The subject may be diagnosed to a have staphylococcal infection, such as a persistant staphylococcal infection or a staphylococcal infection that is resistant to one or more treatments, such as methicillin. The method may further comprise administering to the subject a biological response modifier, such as an adjuvant a cytokine or interleukin. The method may further comprise administering the vaccine more than one time to the subject. The vaccine may be administered orally, parenterally, subcutaneously, intramuscularly, or intravenously. The subject may be a mammal (including a non-human mammal) or a human. The treatment may reduce bacterial load, reduces or prevent renal abscess, or protect a from infection. The method may further comprise provision of a second therapy, such as an antibiotic or antibody as described herein below.

The phrase “does not express a functional SpA, adsA, srtA and/or agrA” is meant to capture all of those mutants that either fail to express any SpA, adsA, srtA and/or agrA, or express reduced amounts or modified forms of these proteins that do not interfere with host immune response. For example a mutation in a regulatory region can reduce the amount of SpA, adsA, srtA and/or agrA to the point where it no longer interferes. Similarly, a mutation that alters the folding of the polypeptide, or that truncates the polypeptide, may serve to block its interfering activity. Alternatively, a more drastic mutation would be the removal or some or all of the relevant coding region from the genome of the bacterium.

An immune response refers to a humoral response, a cellular response, or both a humoral and cellular response in an organism. An immune response can be measured by assays that include, but are not limited to, assays measuring the presence or amount of antibodies that specifically recognize a protein or cell surface protein, assays measuring T-cell activation or proliferation, and/or assays that measure modulation in terms of activity or expression of one or more cytokines.

The SpA, adsA, srtA and/or agrA polypeptides described herein may include 1, 2, 3, 4, 5, 6, 7, 8, 9, 10, 11, 12, 13, 14, 15, or more variant amino acids

In further aspects, a bacterium or vaccine composition may be administered more than one time to the subject, and may be administered 1, 2, 3, 4, 5, 6, 7, 8, 9, 10, 15, 20 or more times. Compositions of the invention are typically administered to human subjects, but administration to other animals that are capable of eliciting an immune response to a staphylococcus bacterium is contemplated, particularly cattle, horses, goats, sheep and other domestic animals, i.e., mammals.

In certain aspects the staphylococcus bacterium is a Staphylococcus aureus. In further embodiments the immune response is a protective immune response. In still further aspects, the methods and compositions of the invention can be used to prevent, ameliorate, reduce, or treat infection of tissues or glands, e.g., mammary glands, particularly mastitis and other infections. Other methods include, but are not limited to prophylactically reducing bacterial burden in a subject not exhibiting signs of infection, particularly those subjects suspected of or at risk of being colonized by a target bacteria, e.g., patients that are or will be at risk or susceptible to infection during a hospital stay, treatment, and/or recovery.

The term “isolated” can refer to a nucleic acid or polypeptide that is substantially free of cellular material, bacterial material, viral material, or culture medium (when produced by recombinant DNA techniques) of their source of origin, or chemical precursors or other chemicals (when chemically synthesized). Moreover, an isolated compound refers to one that can be administered to a subject as an isolated compound; in other words, the compound may not simply be considered “isolated” if it is adhered to a column or embedded in an agarose gel. Moreover, an “isolated nucleic acid fragment” or “isolated peptide” is a nucleic acid or protein fragment that is not naturally occurring as a fragment and/or is not typically in the functional state.

The term “providing” is used according to its ordinary meaning to indicate “to supply or furnish for use.” In some embodiments, the protein is provided directly by administering the protein, while in other embodiments, the protein is effectively provided by administering a nucleic acid that encodes the protein. In certain aspects the invention contemplates compositions comprising various combinations of nucleic acid, antigens, peptides, and/or epitopes.

The subject will have (e.g., are diagnosed with a staphylococcal infection), will be suspected of having, or will be at risk of developing a staphylococcal infection. Compositions of the present invention include immunogenic compositions wherein the antigen(s) or epitope(s) are contained in an amount effective to achieve the intended purpose. More specifically, an effective amount means an amount of active ingredients necessary to stimulate or elicit an immune response, or provide resistance to, amelioration of, or mitigation of infection. In more specific aspects, an effective amount prevents, alleviates or ameliorates symptoms of disease or infection, or prolongs the survival of the subject being treated. Determination of the effective amount is well within the capability of those skilled in the art, especially in light of the detailed disclosure provided herein. For any preparation used in the methods of the invention, an effective amount or dose can be estimated initially from in vitro studies, cell culture, and/or animal model assays. For example, a dose can be formulated in animal models to achieve a desired immune response or circulating antibody concentration or titer. Such information can be used to more accurately determine useful doses in humans.

The embodiments in the Example section are understood to be embodiments of the invention that are applicable to all aspects of the invention.

The use of the term “or” in the claims is used to mean “and/or” unless explicitly indicated to refer to alternatives only or the alternatives are mutually exclusive, although the disclosure supports a definition that refers to only alternatives and “and/or.” It is also contemplated that anything listed using the term “or” may also be specifically excluded.

Throughout this application, the term “about” is used to indicate that a value includes the standard deviation of error for the device or method being employed to determine the value.

Following long-standing patent law, the words “a” and “an,” when used in conjunction with the word “comprising” in the claims or specification, denotes one or more, unless specifically noted.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

So that the matter in which the above-recited features, advantages and objects of the invention as well as others which will become clear are attained and can be understood in detail, more particular descriptions and certain embodiments of the invention briefly summarized above are illustrated in the appended drawings. These drawings form a part of the specification. It is to be noted, however, that the appended drawings illustrate certain embodiments of the invention and therefore are not to be considered limiting in their scope.

FIGS. 1A-K. (FIG. 1A) 4 weeks old BALB/c mice (n=19-20) were infected by intravenous inoculation with 1×10⁷ CFU S. aureus Newman (wild-type), saeR, mgrA, agrA, or srtA transposon insertional mutants. 18 days later, bacterial loads in kidney tissues were examined. Statistical significance was calculated with the unpaired two-tailed Students t-test and P-values recorded; P-values<0.05 were deemed significant. (FIGS. 1B-K) Histopathological evaluation of thin-sectioned, hematoxylin-eosin stained renal tissue was performed to analyze abscess formation. Blue arrows identify staphylococcal abscess communities. White arrows identify PMN infiltrates.

FIGS. 2A-N. (FIG. 2A) 4 weeks old BALB/c mice were infected by intravenous inoculation either with PBS (mock) or 1×10⁷ CFU S. aureus Newman (wild-type), saeR, mgrA, agrA, or srtA transposon insertional mutants. Following antibiotic treatment, mice were challenged by intravenous inoculation with 1×10⁷ CFU S. aureus Newman (wild-type). 4 days later, total bacterial load (TSA) in renal tissues was examined. (FIG. 2B) Staphylococcal burden of the remaining wild-type S. aureus Newman (TSA-TSA/Erm) in renal tissues was examined. Statistical significance was calculated with the unpaired two-tailed Students t-test and P-values recorded; P-values<0.05 were deemed significant. (FIG. 2C-N) Histopathological evaluation of thin-sectioned, hematoxylin-eosin stained renal tissue was performed to identify abscess formation. Blue arrows identify staphylococcal abscess communities. White arrows identify PMN infiltrates.

FIG. 3. 4 weeks old BALB/c mice (n=9-10) were infected by intravenous inoculation with 1×10⁷ CFU S. aureus Newman (wild-type), adsA or spa transposon insertional mutants. 18 days later, bacterial loads in kidney tissues were examined. Statistical significance was calculated with the unpaired two-tailed Students t-test and P-values recorded; P-values<0.05 were deemed significant.

FIGS. 4A-J. (FIG. 4A) 4 weeks old BALB/c mice were infected by intravenous inoculation either with PBS (mock) or 1×10⁷ CFU S. aureus Newman (wild-type), adsA, or spa transposon insertional mutants. Following antibiotic treatment, mice were challenged by intravenous inoculation with 1×10⁷ CFU S. aureus Newman (wild-type). 4 days later, total bacterial loads (TSA) in renal tissues were examined. (FIG. 4B) The remaining burden of S. aureus Newman (TSA-TSA/Erm) in renal tissues was examined. Statistical significance was calculated with the unpaired two-tailed Students t-test and P-values recorded; P-values<0.05 were deemed significant. (FIGS. 4C-J) Histopathological evaluation of thinsectioned, hematoxylin-eosin stained renal tissue was performed to identify abscess formation. Blue arrows identify staphylococcal abscess communities. White arrows identify PMN infiltrates.

FIG. 5. BALB/c mice (n=10) were infected with S. aureus Newman, mgrA or srtA mutant strains for 18 days, and infection cleared with chloramphenicol and ampicillin treatment. Cohorts of animals were terminally bled via cardiac puncture. Immune sera samples were collected and analyzed against the components of the staphylococcal antigen matrix (Table 3). Another cohort of animals was then challenged with S. aureus Newman and bacterial load (log₁₀(CFU)) in kidney tissue homogenate was analyzed after necropsy on day 4. Correlations between bacterial load reduction (mutant strains compare to wild-type) and humoral immune responses toward 27 staphylococcal antigens ([mutant IgG]/[wild-type IgG]) were plotted.

FIGS. 6A-B. BALB/c mice (n=18-20) were either mock immunized with PBS/adjuvant or injected with 25 μg of each antigen (Combo 1, ClfA+SdrD+FnBPB; Combo 2, Combo 1+SpAKKAA). Immunized mice were challenged by intravenous inoculation with 1×10⁷ CFU S. aureus Newman. Bacterial loads in kidney tissues were examined at day 4 (FIG. 6A) and day 18 (FIG. 6B) post-challenge. Statistical significance was calculated with the unpaired two-tailed Students t-test and P-values recorded; P-values<0.05 were deemed significant.

FIG. 7. Virulence defect and protective immunity of strains in renal abscess model.

FIG. 8. Virulence defect and protective immunity of strains in renal abscess model.

FIG. 9. Analysis of hyper-immune sera against staphylococcal antigen matrix.

FIG. 10. Analysis of hyper-immune sera against staphylococcal antigen matrix.

FIG. 11. Analysis of hyper-immune sera against staphylococcal antigen matrix.

FIG. 12. Virulence Defect of S. aureus adsA and spa Variants to form Abscess Lesions in the Kidneys of Infected Mice. BALB/c mice were injected into the retro-orbital plexus with 1×10⁷ CFU of S. aureus Newman (wild-type), adsA or spa variants. Eighteen days after infection, animals were necropsied. Kidneys were analyzed for staphylococcal load as well as histopathology using thin-sectioned, hematoxylin-eosin stained tissue slides. White arrowheads identify polymorphonuclear leukocyte infiltrates. Dark arrowheads identify staphylococcal abscess communities. Animal data are representative of two independent experiments.

FIG. 13. Virulence Defect of S. aureus Newman Variants to Form Abscess Lesions in the Kidneys of Infected Mice. BALB/c mice were injected into the retro-orbital plexus with 1×10⁷ CFU of S. aureus Newman (wild-type), saeR, mgrA, agrA or srtA variants. Four days after infection, animals were necropsied. Kidneys were analyzed for staphylococcal load as well as histopathology using thin-sectioned, hematoxylin-eosin stained tissue slides. White arrowheads identify polymorphonuclear leukocyte infiltrates. Dark arrowheads identify staphylococcal abscess communities. Animal data are representative of two independent experiments.

### DETAILED DESCRIPTION

### I. S. aureus Vaccines

Staphylococcus aureus is a commensal of the human skin and nares, and the leading cause of bloodstream, skin and soft tissue infections (Klevens et al., 2007). Recent dramatic increases in the mortality of staphylococcal diseases are attributed to the spread of methicillin-resistant S. aureus (MRSA) strains often not susceptible to antibiotics (Kennedy et al., 2008). In a large retrospective study, the incidence of MRSA infections was 4.6% of all hospital admissions in the United States (Klevens et al., 2007). The annual health care costs for 94,300 MRSA infected individuals in the United States exceed $2.4 billion (Klevens et al., 2007). The current MRSA epidemic has precipitated a public health crisis that needs to be addressed by development of a preventive vaccine (Boucher and Corey, 2008). To date, an FDA licensed vaccine that prevents S. aureus diseases is not available.

Previously, the inventors demonstrated that infection with virulent S. aureus Newman and clearance of the pathogen with antibiotic treatment did not aid mice in developing protective immunity against subsequent infection with the same strain. Indeed, examination of immune sera did not reveal high amounts of antibodies toward staphylococcal antigens partly due to staphylococcal protein A, a B cell superantigen. Thus, the inventors surmised that the best vaccine antigens would be encoded by genetic determinants also required for the disease process.

Here, the inventors we have examined the foregoing hypothesis that staphylococcal live-attenuated vaccines can elicit protective immunity against subsequent infection with virulent S. aureus, and further, that such immunity results from antibodies against protective antigens. Mutant strains having transposon insertions in saeR, mgrA, and srtA did not persist in animal model, yet had different humoral immune response profiles. Animals infected with srtA mutant generated protective immunity against subsequent infection with the wild-type strain. Among surface molecules anchored by sortase A, AdsA and SpA were previously characterized to modulate innate and humoral immunity. Mutants with insertions into agrA, srtA, adsA and spa all had altered infectivity, but also showed altered ability to induce humoral immune response. Correlation studies between bacterial load reduction and humoral immune responses to 27 staphylococcal antigens indicated that antibodies against ClfA, FnBPB and SdrD can confer protective immunity. These and other aspects of the invention are discussed in detail below.

### II. Staphylococcal Target Proteins

In accordance with the present invention, altered bacteria are provided that lack the ability to express functional or “normal” versions of various proteins, as set out below. These bacteria may be engineered through a number of means, discussed further below, and may include deletion, insertion and trunctation mutants in the genes in question. These altered bacteria have attenuated growth and pathogenicity, but surprisingly produce better immunity that wild-type staphylococcal strains. The following is a discussion of the relevant staphylococcal protein targets.

A. Staphylcoccal Protein A (SpA)

All Staphylococcus aureus strains express the structural gene for Protein A (spa) (Jensen, 1958; Said-Salim et al., 2003), a well characterized virulence factor whose cell wall anchored surface protein product (SpA) encompasses five highly homologous immunoglobulin binding domains designated E, D, A, B, and C (Sjodahl, 1977). These domains display ˜80% identity at the amino acid level, are 56 to 61 residues in length, and are organized as tandem repeats (Uhlen et al., 1984). SpA is synthesized as a precursor protein with an N-terminal YSIRK/GS signal peptide and a C-terminal LPXTG motif sorting signal (DeDent et al., 2008; Schneewind et al., 1992). Cell wall anchored Protein A is displayed in great abundance on the staphylococcal surface (DeDent et al., 2007; Sjoquist et al., 1972). Each of its immunoglobulin binding domains is composed of anti-parallel α-helices that assemble into a three helix bundle and bind the Fc domain of immunoglobulin G (IgG) (Deisenhofer, 1981; Deisenhofer et al., 1978), the VH3 heavy chain (Fab) of IgM (i.e., the B cell receptor) (Graille et al., 2000), the von Willebrand factor at its AI domain [vWF AI is a ligand for platelets] (O'Seaghdha et al., 2006) and the tumor necrosis factor α (TNF-α) receptor I (TNFRI) (Gomez et al., 2006), which is displayed on surfaces of airway epithelia (Gomez et al., 2004; Gomez et al., 2007).

SpA impedes neutrophil phagocytosis of staphylococci through its attribute of binding the Fc component of IgG (Jensen, 1958; Uhlen et al., 1984). Moreover, SpA is able to activate intravascular clotting via its binding to von Willebrand factor AI domains (Hartleib et al., 2000). Plasma proteins such as fibrinogen and fibronectin act as bridges between staphylococci (ClfA and ClfB) and the platelet integrin GPIIb/IIIa (O'Brien et al., 2002), an activity that is supplemented through Protein A association with vWF AI, which allows staphylococci to capture platelets via the GPIb-α platelet receptor (Foster, 2005; O'Seaghdha et al., 2006). SpA also binds TNFRI and this interaction contributes to the pathogenesis of staphylococcal pneumonia (Gomez et al., 2004). SpA activates proinflammatory signaling through TNFR1 mediated activation of TRAF2, the p38/c-Jun kinase, mitogen activate protein kinase (MAPK) and the Rel-transcription factor NF-KB. SpA binding further induces TNFR1 shedding, an activity that appears to require the TNF-converting enzyme (TACE)(Gomez et al., 2007). All of the aforementioned SpA activities are mediated through its five IgG binding domains and can be perturbed by the same amino acid substitutions, initially defined by their requirement for the interaction between Protein A and human IgG1 (Cedergren et al., 1993.

SpA also functions as a B cell superantigen by capturing the Fab region of VH3 bearing IgM, the B cell receptor (Gomez et al., 2007; Goodyear et al., 2003; Goodyear and Silverman, 2004; Roben et al., 1995). Following intravenous challenge, staphylococcal Protein A (SpA) mutations show a reduction in staphylococcal load in organ tissues and dramatically diminished ability to form abscesses (described herein). During infection with wildtype S. aureus, abscesses are formed within forty-eight hours and are detectable by light microscopy of hematoxylin-eosin stained, thin-sectioned kidney tissue, initially marked by an influx of polymorphonuclear leukocytes (PMNs). On day 5 of infection, abscesses increase in size and enclosed a central population of staphylococci, surrounded by a layer of eosinophilic, amorphous material and a large cuff of PMNs. Histopathology revealed massive necrosis of PMNs in proximity to the staphylococcal nidus at the center of abscess lesions as well as a mantle of healthy phagocytes. The inventors also observed a rim of necrotic PMNs at the periphery of abscess lesions, bordering the eosinophilic pseudocapsule that separated healthy renal tissue from the infectious lesion. Staphylococcal variants lacking Protein A are unable to establish the histopathology features of abscesses and are cleared during infection.

In previous studies, Cedergren et al. (1993) engineered five individual substitutions in the Fc fragment binding sub-domain of the B domain of SpA, L17D, N28A, I31A and K35A. These authors created these proteins to test data gathered from a three dimensional structure of a complex between one domain of SpA and Fc₁. Cedergren et al. determined the effects of these mutations on stability and binding, but did not contemplate use of such substitutions for the production of a vaccine antigen.

Brown et al. (1998) describe studies designed to engineer new proteins based on SpA that allow the use of more favorable elution conditions when used as affinity ligands. The mutations studied included single mutations of Q13A, Q14H, N15A, N15H, F17H, Y18F, L21H, N32H, or K39H. Brown et al. report that Q13A, N15A, N15H, and N32H substitutions made little difference to the dissociation constant values and that the Y18F substitution resulted in a 2 fold decrease in binding affinity as compared to wild type SpA. Brown et al. also report that L21H and F17H substitutions decrease the binding affinity by five-fold and a hundred-fold respectively. The authors also studied analogous substitutions in two tandem domains. Thus, the Brown et al. studies were directed to generating a SpA with a more favorable elution profile, hence the use of His substitutions to provide a pH sensitive alteration in the binding affinity. Brown et al. is silent on the use of SpA as a vaccine antigen.

Graille et al. (2000) describe a crystal structure of domain D of SpA and the Fab fragment of a human IgM antibody. Graille et al. define by analysis of a crystal structure the D domain amino acid residues that interact with the Fab fragment as residues Q26, G29, F30, Q32, S33, D36, D37, Q40, N43, E47, or L51, as well as the amino acid residues that form the interface between the domain D sub-domains. Graille et al. define the molecular interactions of these two proteins, but is silent in regard to any use of substitutions in the interacting residues in producing a vaccine antigen.

O'Seaghdha et al. (2006) describe studies directed at elucidating which sub-domain of domain D binds vWF. The authors generated single mutations in either the Fc or VH3 binding sub-domains, i.e., amino acid residues F5A, Q9A, Q10A, F13A, Y14A, L17A, N28A, I31A, K35A, G29A, F30A, S33A, D36A, D37A, Q40A, E47A, or Q32A. The authors discovered that vWF binds the same sub-domain that binds Fc. O'Seaghda et al. define the sub-domain of domain D responsible for binding vWF, but is silent in regard to any use of substitutions in the interacting residues in producing a vaccine antigen.

Gomez et al. (2006) describe the identification of residues responsible for activation of the TNFR1 by using single mutations of F5A, F13A, Y14A, L17A, N21A, I31A, Q32A, and K35A. Gomez et al. is silent in regard to any use of substitutions in the interacting residues in producing a vaccine antigen.

Recombinant affinity tagged Protein A, a polypeptide encompassing the five IgG domains (EDCAB) (Sjodahl, 1977) but lacking the C-terminal Region X (Guss et al., 1984), was purified from recombinant E. coli and used as a vaccine antigen (Stranger-Jones et al., 2006). Because of the attributes of SpA in binding the Fc portion of IgG, a specific humoral immune response to Protein A could not be measured (Stranger-Jones et al., 2006). The inventors have overcome this obstacle through the generation of SpA-DQ9,10K;D36,37A. BALB/c mice immunized with recombinant Protein A (SpA) displayed significant protection against intravenous challenge with S. aureus strains: a 2.951 log reduction in staphylococcal load as compared to the wild-type (P>0.005; Student's t-test) (Stranger-Jones et al., 2006). SpA specific antibodies may cause phagocytic clearance prior to abscess formation and/or impact the formation of the aforementioned eosinophilic barrier in abscesses that separate staphylococcal communities from immune cells since these do not form during infection with Protein A mutant strains. Each of the five SpA domains (i.e., domains formed from three helix bundles designated E, D, A, B, and C) exerts similar binding properties (Jansson et al., 1998). The solution and crystal structure of the domain D has been solved both with and without the Fc and VH3 (Fab) ligands, which bind Protein A in a non-competitive manner at distinct sites (Graille et al., 2000). Mutations in residues known to be involved in IgG binding (FS, Q9, Q10, S11, F13, Y14, L17, N28, I31 and K35) are also required for vWF AI and TNFR1 binding (Cedergren et al., 1993; Gomez et al., 2006; O'Seaghdha et al., 2006), whereas residues important for the VH3 interaction (Q26, G29, F30, S33, D36, D37, Q40, N43, E47) appear to have no impact on the other binding activities (Graille et al., 2000; Jansson et al., 1998). SpA specifically targets a subset of B cells that express VH3 family related IgM on their surface, i.e., VH3 type B cell receptors (Roben et al., 1995). Upon interaction with SpA, these B cells proliferate and commit to apoptosis, leading to preferential and prolonged deletion of innate-like B lymphocytes (i.e., marginal zone B cells and follicular B2 cells) (Goodyear et al., 2003; Goodyear et al., 2004).

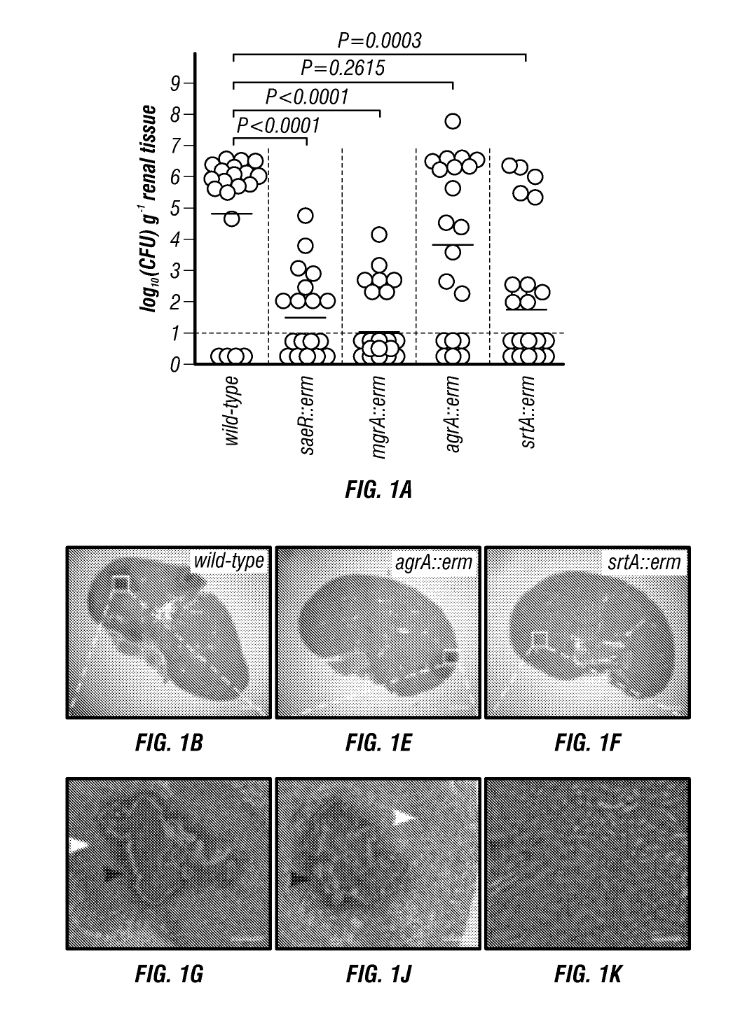

Protein A is synthesized as a precursor in the bacterial cytoplasm and secreted via its YSIRK signal peptide at the cross wall, i.e. the cell division septum of staphylococci (FIG. 1) (DeDent et al., 2007; DeDent et al., 2008). Following cleavage of the C-terminal LPXTG sorting signal, Protein A is anchored to bacterial peptidoglycan crossbridges by sortase A (Mazmanian et al., 1999; Schneewind et al., 1995; Mazmanian et al., 2000). Protein A is the most abundant surface protein of staphylococci; the molecule is expressed by virtually all S. aureus strains (Cespedes et al., 2005; Kennedy et al., 2008; Said-Salim et al., 2003). Staphylococci turn over 15-20% of their cell wall per division cycle (Navarre and Schneewind, 1999). Murine hydrolases cleave the glycan strands and wall peptides of peptidoglycan, thereby releasing Protein A with its attached C-terminal cell wall disaccharide tetrapeptide into the extracellular medium (Ton-That et al., 1999). Thus, by physiological design, Protein A is both anchored to the cell wall and displayed on the bacterial surface but also released into surrounding tissues during host infection (Marraffini et al., 2006).

Protein A captures immunoglobulins on the bacterial surface and this biochemical activity enables staphylococcal escape from host innate and acquired immune responses (Jensen, 1958; Goodyear et al., 2004). Interestingly, region X of Protein A (Guss et al., 1984), a repeat domain that tethers the IgG binding domains to the LPXTG sorting signal /cell wall anchor, is perhaps the most variable portion of the staphylococcal genome (Said-Salim, 2003; Schneewind et al., 1992). Each of the five immunoglobulin binding domains of Protein A (SpA), formed from three helix bundles and designated E, D, A, B, and C, exerts similar structural and functional properties (Sjodahl, 1977; Jansson et al., 1998). The solution and crystal structure of the domain D has been solved both with and without the Fc and V_{H}3 (Fab) ligands, which bind Protein A in a non-competitive manner at distinct sites (Graille 2000).

In the crystal structure complex, the Fab interacts with helix II and helix III of domain D via a surface composed of four VH region β-strands (Graille 2000). The major axis of helix II of domain D is approximately 50° to the orientation of the strands, and the interhelical portion of domain D is most proximal to the CO strand. The site of interaction on Fab is remote from the Ig light chain and the heavy chain constant region. The interaction involves the following domain D residues: Asp-36 of helix II, Asp-37 and Gln-40 in the loop between helix II and helix III and several other residues (Graille 2000). Both interacting surfaces are composed predominantly of polar side chains, with three negatively charged residues on domain D and two positively charged residues on the 2A2 Fab buried by the interaction, providing an overall electrostatic attraction between the two molecules. Of the five polar interactions identified between Fab and domain D, three are between side chains. A salt bridge is formed between Arg-H19 and Asp-36 and two hydrogen bonds are made between Tyr-H59 and Asp-37 and between Asn-H82a and Ser-33. Because of the conservation of Asp-36 and Asp-37 in all five IgG binding domains of Protein A, the inventors mutated these residues.

The SpA-D sites responsible for Fab binding are structurally separate from the domain surface that mediates Fcγ binding. The interaction of Fcγ with domain D primarily involves residues in helix I with lesser involvement of helix II (Gouda et al., 1992; Deisenhofer, 1981). With the exception of the Gln-32, a minor contact in both complexes, none of the residues that mediate the Fcγ interaction are involved in Fab binding. To examine the spatial relationship between these different Ig-binding sites, the SpA domains in these complexes have been superimposed to construct a model of a complex between Fab, the SpA-domain D, and the Fcγ molecule. In this ternary model, Fab and Fcγ form a sandwich about opposite faces of the helix II without evidence of steric hindrance of either interaction. These findings illustrate how, despite its small size (i.e., 56-61 aa), an SpA domain can simultaneously display both activities, explaining experimental evidence that the interactions of Fab with an individual domain are noncompetitive. Residues for the interaction between SpA-D and Fcγ are Gln-9 and Gln-10.

In contrast, occupancy of the Fc portion of IgG on the domain D blocks its interaction with vWF AI and probably also TNFR1 (O'Seaghdha et al., 2006). Mutations in residues essential for IgG Fc binding (F5, Q9, Q10, S11, F13, Y14, L17, N28, I31 and K35) are also required for vWF AI and TNFR1 binding (O'Seaghdha et al., 2006; Cedergren et al., 1993; Gomez et al., 2006), whereas residues critical for the VH3 interaction (Q26, G29, F30, S33, D36, D37, Q40, N43, E47) have no impact on the binding activities of IgG Fc, vWF AI or TNFR1 (Jansson et al., 1998; Graille et al., 2000). The Protein A immunoglobulin Fab binding activity targets a subset of B cells that express V_{H}3 family related IgM on their surface, i.e., these molecules function as VH3type B cell receptors (Roben et al., 1995). Upon interaction with SpA, these B cells rapidly proliferate and then commit to apoptosis, leading to preferential and prolonged deletion of innate-like B lymphocytes (i.e., marginal zone B cells and follicular B2 cells) (Goodyear and Silverman, 2004; Goodyear and Silverman, 2003). More than 40% of circulating B cells are targeted by the Protein A interaction and the V_{H}3 family represents the largest family of human B cell receptors to impart protective humoral responses against pathogens (Goodyear and Silverman, 2004; Goodyear and Silverman, 2003). Thus, Protein A functions analogously to staphylococcal superantigens (Roben et al., 1995), albeit that the latter class of molecules, for example SEB, TSST-1, TSST-2, form complexes with the T cell receptor to inappropriately stimulate host immune responses and thereby precipitating characteristic disease features of staphylococcal infections (Roben et al., 1995; Tiedemann et al., 1995). Together these findings document the contributions of Protein A in establishing staphylococcal infections and in modulating host immune responses.

In sum, Protein A domains can viewed as displaying two different interfaces for binding with host molecules and any development of Protein A based vaccines must consider the generation of variants that do not perturb host cell signaling, platelet aggregation, sequestration of immunoglobulins or the induction of B cell proliferation and apoptosis. Such Protein A variants should also be useful in analyzing vaccines for the ability of raising antibodies that block the aforementioned SpA activities and occupy the five repeat domains at their dual binding interfaces.

B. Staphylococcal agrA

The agr locus encodes the components of an autoregulatory quorum-sensing system that controls expression of the regulatory RNA molecule RNAIII. Components of this system include agrD, the signaling peptide; agrB, the secretory protein responsible for the export and processing of agrD to its active form; and agrC/agrA, a two-component histidine kinase and response regulator system that detects agrD at critical levels and initiates the expression of those virulence determinants under agr control.

agrA is one member of a family of conserved response regulators with CheY-like receiver domains. These response regulators undergo conformational changes upon the phosphorylation of an aspartate residue by the cognate sensory histidine kinase, allowing them to bind to promoter elements and upregulate transcription. agrA 238 amino acid protein (accession for S. aureus strain Newman is YP_{—}001332980; SEQ ID NO:2) of the LytR family of response regulators that recognize a novel element consisting of a pair of direct repeats having a consensus sequence of (TA)([AC)(CA)GTTN(AG)(TG), and separated by a 12- to 13-bp spacer region. Two such elements are found in the P2-P3 intergenic region of RNAIII and the agr operon.

Whereas the agr two-component system has been assumed to follow the canonical quorum-sensing model, the inability to demonstrate binding of agrA to the RNAIII-agr intergenic region led some researchers to question the identification of agrA as a DNA-binding response regulator. However, using purified recombinant agrA in electrophoretic mobility shift assays (EMSAs), agrA has been shown to bind to the P2-P3 region of the agr locus with high affinity. The strongest binding was found to be localized to the pair of direct repeats in the P2 promoter region, with binding to the corresponding pair of repeats in the P3 promoter region being weaker. Phosphorylation of agrA by small phosphodonors had differential effects on binding affinity at the two sites.

C. Staphylococcal srtA

Staphylococcal srtA (surface protein sorting A) is a 206 amino acid polypeptide with an N-terminal hydrophobic domain that functions as a signal peptide/membrane anchor domain. Studies suggest that srtA is assembled in the membrane envelope as a type II membrane protein with its N-terminus in the cytoplasm and the C-terminal end positioned in the cell wall. Strains mutated in srtA are defective in cleaving the sorting signals of protein, fibronectin binding proteins A and B, and clumping factor. As such, srtA is necessary for the cell wall anchoring of certain surface proteins. The accession number for S. aureus Newman srtA is YP_{—}001333460 (SEQ ID NO:1).

D. Staphylococcal adsA

Adenosine synthase A (adsA), a cell wall-anchored enzyme that converts adenosine monophosphate to adenosine, as a critical virulence factor. Staphylococcal synthesis of adenosine in blood, escape from phagocytic clearance, and subsequent formation of organ abscesses are all dependent on adsA and can be rescued by an exogenous supply of adenosine. adsA homologues exist in anthrax and Bacillus anthracis where it protects from phagocytic clearance. Clearly, staphylococci and other bacterial pathogens exploit the immunomodulatory attributes of adenosine, through adsA, to escape host immune responses.

E. Proteins

The sequences of any of the above proteins may vary from strain to strain and between Staphylococcal species. However, those of skill in the art can identify the corresponding proteins and genes by homology. Also, the term “functionally equivalent codon” is used herein to refer to codons that encode the same amino acid, such as the six codons for arginine or serine, and also refers to codons that encode biologically equivalent amino acids (see Table 1, below). This degeneracy allows variation in nucleic acid sequences when proteins are identical.

TABLE 1
Codon Table

| Amino Acids | | | Codons |
|---|---|---|---|
| Alanine | Ala | A | GCA GCC GCG GCU |
| Cysteine | Cys | C | UGC UGU |
| Aspartic | Asp | D | GAC GAU |
| acid | | | |
| Glutamic | Glu | E | GAA GAG |
| acid | | | |
| Phenylalanine | Phe | F | UUC UUU |
| Glycine | Gly | G | GGA GGC GGG GGU |
| Histidine | His | H | CAC CAU |
| Isoleucine | Ile | I | AUA AUC AUU |
| Lysine | Lys | K | AAA AAG |
| Leucine | Leu | L | UUA UUG CUA CUC CUG CUU |
| Methionine | Met | M | AUG |
| Asparagine | Asn | N | AAC AAU |
| Proline | Pro | P | CCA CCC CCG CCU |
| Glutamine | Gln | Q | CAA CAG |
| Arginine | Arg | R | AGA AGG CGA CGC CGG CGU |
| Serine | Ser | S | AGC AGU UCA UCC UCG UCU |
| Threonine | Thr | T | ACA ACC ACG ACU |
| Valine | Val | V | GUA GUC GUG GUU |
| Tryptophan | Trp | W | UGG |
| Tyrosine | Tyr | Y | UAC UAU |

It also will be understood that proteins and genes may include additional residues, such as additional N- or C-terminal amino acids, or 5′ or 3′ sequences, respectively, natural or synthetic, and yet still be essentially as set forth in one of the proteins disclosed herein, so long as the sequence meets the criteria set forth above, including the maintenance of biological protein activity (e.g., immunogenicity) where protein expression is concerned. The addition of terminal sequences particularly applies to nucleic acid sequences that may, for example, include various non-coding sequences flanking either of the 5′ or 3′ portions of the coding region.

### II. Nucleic Acids

In certain embodiments, the present invention concerns recombinant polynucleotides encoding for producing, and also encoding, attenuated bacteria of the invention. The nucleic acid sequences for adsA, srtA, agrA and SpA, along with entire genomic sequences are well known to those in the art. The entire sequence for S. aureus Newman is at accession no. NC_{—}009641.

As used in this application, the term “polynucleotide” refers to a nucleic acid molecule that either is recombinant or has been isolated free of total genomic nucleic acid. Included within the term “polynucleotide” are oligonucleotides (nucleic acids of 100 residues or less in length), recombinant vectors, including, for example, plasmids, cosmids, phage, viruses, and the like. Polynucleotides include, in certain aspects, regulatory sequences, isolated substantially away from their naturally occurring genes or protein encoding sequences. Polynucleotides may be single-stranded (coding or antisense) or double-stranded, and may be RNA, DNA (genomic, cDNA or synthetic), analogs thereof, or a combination thereof. Additional coding or non-coding sequences may, but need not, be present within a polynucleotide.

In this respect, the term “gene,” “polynucleotide,” or “nucleic acid” is used to refer to a nucleic acid that encodes a protein, polypeptide, or peptide (including any sequences required for proper transcription, post-translational modification, or localization). As will be understood by those in the art, this term encompasses genomic sequences, expression cassettes, cDNA sequences, and smaller engineered nucleic acid segments that express, or may be adapted to express, proteins, polypeptides, domains, peptides, fusion proteins, and mutants. A nucleic acid encoding all or part of a polypeptide may contain a contiguous nucleic acid sequence of: 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1010, 1020, 1030, 1040, 1050, 1060, 1070, 1080, 1090, 1095, 1100, 1500, 2000, 2500, 3000, 3500, 4000, 4500, 5000, 5500, 6000, 6500, 7000, 7500, 8000, 9000, 10000, or more nucleotides, nucleosides, or base pairs, including all values and ranges therebetween, of a polynucleotide encoding one or more amino acid sequence described or referenced herein. It also is contemplated that a particular polypeptide may be encoded by nucleic acids containing variations having slightly different nucleic acid sequences but, nonetheless, encode the same or substantially similar protein.

The nucleic acid segments used in the present invention can be combined with other nucleic acid sequences, such as promoters, polyadenylation signals, additional restriction enzyme sites, multiple cloning sites, other coding segments, and the like, such that their overall length may vary considerably. It is therefore contemplated that a nucleic acid fragment of almost any length may be employed, with the total length preferably being limited by the ease of preparation and use in the intended recombinant nucleic acid protocol. In some cases, a nucleic acid sequence may encode a polypeptide sequence with additional heterologous coding sequences, for example to allow for purification of the polypeptide, transport, secretion, post-translational modification, or for therapeutic benefits such as targeting or efficacy. As discussed above, a tag or other heterologous polypeptide may be added to the modified polypeptide-encoding sequence, wherein “heterologous” refers to a polypeptide that is not the same as the modified polypeptide.

In certain other embodiments, the invention concerns isolated nucleic acid segments and recombinant vectors that include within their sequence a contiguous nucleic acid sequence from SEQ ID NO:1 or SEQ ID NO:2 or any other nucleic acid sequences encoding target proteins.

In certain embodiments, the present invention provides polynucleotide variants having substantial identity to the sequences disclosed herein; those comprising at least 70%, 75%, 80%, 85%, 90%, 95%, 96%, 97%, 98%, or 99% or higher sequence identity, including all values and ranges there between, compared to a polynucleotide sequence of this invention using the methods described herein (e.g., BLAST analysis using standard parameters).

A. Vectors

The term “vector” is used to refer to a carrier nucleic acid molecule into which a heterologous nucleic acid sequence can be inserted. A nucleic acid sequence can be “heterologous,” which means that it is in a context foreign to the cell in which the vector is being introduced or to the nucleic acid in which is incorporated, which includes a sequence homologous to a sequence in the cell or nucleic acid but in a position within the host cell or nucleic acid where it is ordinarily not found. Vectors include DNAs, RNAs, plasmids, cosmids, viruses (bacteriophage, animal viruses, and plant viruses), and artificial chromosomes (e.g., YACs). One of skill in the art would be well equipped to construct a vector through standard recombinant techniques (for example Sambrook et al., 2001; Ausubel et al., 1996, both incorporated herein by reference). Useful vectors encoding such fusion proteins include pIN vectors (Inouye et al., 1985), vectors encoding a stretch of histidines, and pGEX vectors, for use in generating glutathione S-transferase (GST) soluble fusion proteins for later purification and separation or cleavage. A particular vector in accordance with the present invention is one that carries a transposon.

The term “expression vector” refers to a vector containing a nucleic acid sequence coding for at least part of a gene product capable of being transcribed. In some cases, RNA molecules are then translated into a protein, polypeptide, or peptide. Expression vectors can contain a variety of “control sequences,” which refer to nucleic acid sequences necessary for the transcription and possibly translation of an operably linked coding sequence in a particular host organism. In addition to control sequences that govern transcription and translation, vectors and expression vectors may contain nucleic acid sequences that serve other functions as well and are described herein.

1. Promoters and Enhancers

A “promoter” is a control sequence. The promoter is typically a region of a nucleic acid sequence at which initiation and rate of transcription are controlled. It may contain genetic elements at which regulatory proteins and molecules may bind such as RNA polymerase and other transcription factors. The phrases “operatively positioned,” “operatively linked,” “under control,” and “under transcriptional control” mean that a promoter is in a correct functional location and/or orientation in relation to a nucleic acid sequence to control transcriptional initiation and expression of that sequence. A promoter may or may not be used in conjunction with an “enhancer,” which refers to a cis-acting regulatory sequence involved in the transcriptional activation of a nucleic acid sequence.

Naturally, it may be important to employ a promoter and/or enhancer that effectively directs the expression of the DNA segment in the cell type or organism chosen for expression. Those of skill in the art of molecular biology generally know the use of promoters, enhancers, and cell type combinations for protein expression (see Sambrook et al., 2001, incorporated herein by reference). The promoters employed may be constitutive, tissue-specific, or inducible and in certain embodiments may direct high level expression of the introduced DNA segment under specified conditions, such as large-scale production of recombinant proteins or peptides.

The particular promoter that is employed to control the expression of peptide or protein encoding polynucleotide of the invention is not believed to be critical, so long as it is capable of expressing the polynucleotide in a targeted cell, preferably a bacterial cell. Where a human cell is targeted, it is preferable to position the polynucleotide coding region adjacent to and under the control of a promoter that is capable of being expressed in a human cell. Generally speaking, such a promoter might include either a bacterial, human or viral promoter.

2. Initiation Signals and Internal Ribosome Binding Sites (IRES)

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals.

In certain embodiments of the invention, the use of internal ribosome entry sites (IRES) elements are used to create multigene, or polycistronic, messages. IRES elements are able to bypass the ribosome scanning model of 5′-methylated Cap dependent translation and begin translation at internal sites (Pelletier and Sonenberg, 1988; Macejak and Sarnow, 1991). IRES elements can be linked to heterologous open reading frames. Multiple open reading frames can be transcribed together, each separated by an IRES, creating polycistronic messages. Multiple genes can be efficiently expressed using a single promoter/enhancer to transcribe a single message (see U.S. Pat. Nos. 5,925,565 and 5,935,819, herein incorporated by reference).

3. Selectable and Screenable Markers

In certain embodiments of the invention, cells containing a nucleic acid construct of the present invention may be identified in vitro or in vivo by encoding a screenable or selectable marker in the expression vector. When transcribed and translated, a marker confers an identifiable change to the cell permitting easy identification of cells containing the expression vector. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker.

Of particular interest are markers that create drug sensitivity in the engineered bacteria of the present invention, such as antibiotic markers. While it is viewed that the attenuated strains of the present invention will be safe for use in subjects, the ability to specifically inhibit these vaccine strains is a useful tool. Various antibiotic resistance markers are well known to those in the art.

B. Host Cells

As used herein, the terms “cell,” “cell line,” and “cell culture” may be used interchangeably. All of these terms also include their progeny, which is any and all subsequent generations. It is understood that all progeny may not be identical due to deliberate or inadvertent mutations. In the context of expressing a heterologous nucleic acid sequence, “host cell” refers to a prokaryotic or eukaryotic cell, and it includes any transformable organism that is capable of replicating a vector or expressing a heterologous gene encoded by a vector. A host cell can, and has been, used as a recipient for vectors or viruses. A host cell may be “transfected” or “transformed,” which refers to a process by which exogenous nucleic acid, such as a recombinant protein-encoding sequence, is transferred or introduced into the host cell. A transformed cell includes the primary subject cell and its progeny.

Host cells may be derived from prokaryotes or eukaryotes, including bacteria, yeast cells, insect cells, and mammalian cells for replication of the vector or expression of part or all of the nucleic acid sequence(s). Numerous cell lines and cultures are available for use as a host cell, and they can be obtained through the American Type Culture Collection (ATCC), which is an organization that serves as an archive for living cultures and genetic materials (World Wide Web at atcc.org).

C. Mutagenic Procedures

Transposable elements are an important source of spontaneous mutations, and have influenced the ways in which genes and genomes have evolved. They can inactivate genes by inserting within them, and can cause gross chromosomal rearrangements either directly, through the activity of their transposases, or indirectly, as a result of recombination between copies of an element scattered around the genome. Transposable elements that excise often do so imprecisely and may produce alleles coding for altered gene products if the number of bases added or deleted is a multiple of three. Transposable elements can also be used to “knock in” heterologous sequences.

Transposable elements themselves may evolve in unusual ways. If they were inherited like other DNA sequences, then copies of an element in one species would be more like copies in closely related species than copies in more distant species. This is not always the case, suggesting that transposable elements are occasionally transmitted horizontally from one species to another. In accordance with the present invention, mutations will be introduced into gram-positive bacteria such as S. aureus using a Himar 1 transposase.

Himar 1 is a “mariner,” one of a widespread and diverse family of animal transposons. Himar 1 is derived from Haematobia irritans. This transposase can reproduce transposition faithfully in an in vitro inter-plasmid transposition reaction. It binds to the inverted terminal repeat sequences of its cognate transposon and mediates 5′ and 3′ cleavage of the element termini. It functions independent of species-specific host factors, which explains the broad distribution of mariners and why they are capable of horizontal transfer between species (Lampe et al., 1996).

U.S. Patent Application Publication No. 2006/0275905 also discloses suitable mutagenic procedures and is hereby incorporated by reference.

### IV. Immune Response and Assays

As discussed above, the invention concerns evoking or inducing an immune response in a subject. In one embodiment, the immune response can protect against or treat a subject having, suspected of having, or at risk of developing an infection or related disease, particularly those related to staphylococci. One use of the immunogenic compositions of the invention is to prevent nosocomial infections by inoculating a subject prior to undergoing procedures in a hospital or other environment having an increased risk of infection.

Staphylococcal infections progress through several different stages. For example, the staphylococcal life cycle involves commensal colonization, initiation of infection by accessing adjoining tissues or the bloodstream, and/or anaerobic multiplication in the blood. The interplay between S. aureus virulence determinants and the host defense mechanisms can induce complications such as endocarditis, metastatic abscess formation, and sepsis syndrome. Different molecules on the surface of the bacterium are involved in different steps of the infection cycle. Combinations of certain antigens can elicit an immune response which protects against multiple stages of staphylococcal infection. The effectiveness of the immune response can be measured either in animal model assays and/or using an opsonophagocytic assay.

A. Immunoassays

The present invention includes the implementation of serological assays to evaluate whether and to what extent an immune response is induced or evoked by compositions of the invention. There are many types of immunoassays that can be implemented. Immunoassays encompassed by the present invention include, but are not limited to, those described in U.S. Pat. No. 4,367,110 (double monoclonal antibody sandwich assay) and U.S. Pat. No. 4,452,901 (western blot). Other assays include immunoprecipitation of labeled ligands and immunocytochemistry, both in vitro and in vivo.

Immunoassays generally are binding assays. Certain preferred immunoassays are the various types of enzyme linked immunosorbent assays (ELISAs) and radioimmunoassays (RIA) known in the art. Immunohistochemical detection using tissue sections is also particularly useful. In one example, antibodies or antigens are immobilized on a selected surface, such as a well in a polystyrene microtiter plate, dipstick, or column support. Then, a test composition suspected of containing the desired antigen or antibody, such as a clinical sample, is added to the wells. After binding and washing to remove non specifically bound immune complexes, the bound antigen or antibody may be detected. Detection is generally achieved by the addition of another antibody, specific for the desired antigen or antibody, that is linked to a detectable label. This type of ELISA is known as a “sandwich ELISA.” Detection also may be achieved by the addition of a second antibody specific for the desired antigen, followed by the addition of a third antibody that has binding affinity for the second antibody, with the third antibody being linked to a detectable label.

Competition ELISAs are also possible implementations in which test samples compete for binding with known amounts of labeled antigens or antibodies. The amount of reactive species in the unknown sample is determined by mixing the sample with the known labeled species before or during incubation with coated wells. The presence of reactive species in the sample acts to reduce the amount of labeled species available for binding to the well and thus reduces the ultimate signal. Irrespective of the format employed, ELISAs have certain features in common, such as coating, incubating or binding, washing to remove non specifically bound species, and detecting the bound immune complexes.

Antigen or antibodies may also be linked to a solid support, such as in the form of plate, beads, dipstick, membrane, or column matrix, and the sample to be analyzed is applied to the immobilized antigen or antibody. In coating a plate with either antigen or antibody, one will generally incubate the wells of the plate with a solution of the antigen or antibody, either overnight or for a specified period. The wells of the plate will then be washed to remove incompletely-adsorbed material. Any remaining available surfaces of the wells are then “coated” with a nonspecific protein that is antigenically neutral with regard to the test antisera. These include bovine serum albumin (BSA), casein, and solutions of milk powder. The coating allows for blocking of nonspecific adsorption sites on the immobilizing surface and thus reduces the background caused by nonspecific binding of antisera onto the surface.

B. Diagnosis of Bacterial Infection

In addition to the use of proteins, polypeptides, and/or peptides, as well as antibodies binding these polypeptides, proteins, and/or peptides, to treat or prevent infection as described above, the present invention contemplates the use of these polypeptides, proteins, peptides, and/or antibodies in a variety of ways, including the detection of the presence of staphylococcus to diagnose an infection, whether in a patient or on medical equipment which may also become infected. In accordance with the invention, a preferred method of detecting the presence of infections involves the steps of obtaining a sample suspected of being infected by one or more staphylococcal bacteria species or strains, such as a sample taken from an individual, for example, from one's blood, saliva, tissues, bone, muscle, cartilage, or skin. Following isolation of the sample, diagnostic assays utilizing the polypeptides, proteins, peptides, and/or antibodies of the present invention may be carried out to detect the presence of staphylococci, and such assay techniques for determining such presence in a sample are well known to those skilled in the art and include methods such as radioimmunoassay, western blot analysis and ELISA assays. In general, in accordance with the invention, a method of diagnosing an infection is contemplated wherein a sample suspected of being infected with staphylococci has added to it the polypeptide, protein, peptide, antibody, or monoclonal antibody in accordance with the present invention, and staphylococci are indicated by antibody binding to the polypeptides, proteins, and/or peptides, or polypeptides, proteins, and/or peptides binding to the antibodies in the sample.

Accordingly, antibodies produced in accordance with the invention may be used for the prevention of infection from staphylococcal bacteria (i.e., passive immunization), for the treatment of an ongoing infection, or for use as research tools. The term “antibodies” as used herein includes monoclonal, polyclonal, chimeric, single chain, bispecific, simianized, and humanized or primatized antibodies as well as Fab fragments, such as those fragments which maintain the binding specificity of the antibodies, including the products of an Fab immunoglobulin expression library. Accordingly, the invention contemplates the use of single chains such as the variable heavy and light chains of the antibodies. Generation of any of these types of antibodies or antibody fragments is well known to those skilled in the art. Specific examples of the generation of an antibody to a bacterial protein can be found in U.S. Patent Application Pub. No. 20030153022, which is incorporated herein by reference in its entirety.

C. Protective Immunity

In some embodiments of the invention, proteinaceous compositions confer protective immunity to a subject. Protective immunity refers to a body's ability to mount a specific immune response that protects the subject from developing a particular disease or condition that involves the agent against which there is an immune response. An immunogenically effective amount is capable of conferring protective immunity to the subject.

As used herein the phrase “immune response” or its equivalent “immunological response” refers to the development of a humoral (antibody mediated), cellular (mediated by antigen-specific T cells or their secretion products) or both humoral and cellular response directed against a protein, peptide, carbohydrate, or polypeptide of the invention in a recipient patient. Such a response can be an active response induced by administration of immunogen or a passive response induced by administration of antibody, antibody containing material, or primed T-cells. A cellular immune response is elicited by the presentation of polypeptide epitopes in association with Class I or Class II MHC molecules, to activate antigen-specific CD4 (+) T helper cells and/or CD8 (+) cytotoxic T cells. The response may also involve activation of monocytes, macrophages, NK cells, basophils, dendritic cells, astrocytes, microglia cells, eosinophils or other components of innate immunity. As used herein “active immunity” refers to any immunity conferred upon a subject by administration of an antigen.

As used herein “passive immunity” refers to any immunity conferred upon a subject without administration of an antigen to the subject. “Passive immunity” therefore includes, but is not limited to, administration of activated immune effectors including cellular mediators or protein mediators (e.g., monoclonal and/or polyclonal antibodies) of an immune response. A monoclonal or polyclonal antibody composition may be used in passive immunization for the prevention or treatment of infection by organisms that carry the antigen recognized by the antibody. An antibody composition may include antibodies that bind to a variety of antigens that may in turn be associated with various organisms. The antibody component can be a polyclonal antiserum. In certain aspects the antibody or antibodies are affinity purified from an animal or second subject that has been challenged with an antigen(s). Alternatively, an antibody mixture may be used, which is a mixture of monoclonal and/or polyclonal antibodies to antigens present in the same, related, or different microbes or organisms, such as gram-positive bacteria, gram-negative bacteria, including but not limited to staphylococcus bacteria.

Passive immunity may be imparted to a patient or subject by administering to the patient immunoglobulins (Ig) and/or other immune factors obtained from a donor or other non-patient source having a known immunoreactivity. In other aspects, an antigenic composition of the present invention can be administered to a subject who then acts as a source or donor for globulin, produced in response to challenge with the antigenic composition (“hyperimmune globulin”), that contains antibodies directed against Staphylococcus or other organism. A subject thus treated would donate plasma from which hyperimmune globulin would then be obtained, via conventional plasma-fractionation methodology, and administered to another subject in order to impart resistance against or to treat staphylococcus infection. Hyperimmune globulins according to the invention are particularly useful for immune-compromised individuals, for individuals undergoing invasive procedures or where time does not permit the individual to produce their own antibodies in response to vaccination. See U.S. Pat. Nos. 6,936,258, 6,770,278, 6,756,361, 5,548,066, 5,512,282, 4,338,298, and 4,748,018, each of which is incorporated herein by reference in its entirety, for exemplary methods and compositions related to passive immunity.

For purposes of this specification and the accompanying claims the terms “epitope” and “antigenic determinant” are used interchangeably to refer to a site on an antigen to which B and/or T cells respond or recognize. B-cell epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance. See, e.g., Epitope Mapping Protocols (1996). Antibodies that recognize the same epitope can be identified in a simple immunoassay showing the ability of one antibody to block the binding of another antibody to a target antigen. T-cells recognize continuous epitopes of about nine amino acids for CD8 cells or about 13-15 amino acids for CD4 cells. T cells that recognize the epitope can be identified by in vitro assays that measure antigen-dependent proliferation, as determined by ³H-thymidine incorporation by primed T cells in response to an epitope (Burke et al., 1994), by antigen-dependent killing (cytotoxic T lymphocyte assay, Tigges et al., 1996) or by cytokine secretion.

The presence of a cell-mediated immunological response can be determined by proliferation assays (CD4 (+) T cells) or CTL (cytotoxic T lymphocyte) assays. The relative contributions of humoral and cellular responses to the protective or therapeutic effect of an immunogen can be distinguished by separately isolating IgG and T-cells from an immunized syngeneic animal and measuring protective or therapeutic effect in a second subject.

As used herein and in the claims, the terms “antibody” or “immunoglobulin” are used interchangeably and refer to any of several classes of structurally related proteins that function as part of the immune response of an animal or recipient, which proteins include IgG, IgD, IgE, IgA, IgM and related proteins.

Under normal physiological conditions antibodies are found in plasma and other body fluids and in the membrane of certain cells and are produced by lymphocytes of the type denoted B cells or their functional equivalent. Antibodies of the IgG class are made up of four polypeptide chains linked together by disulfide bonds. The four chains of intact IgG molecules are two identical heavy chains referred to as H-chains and two identical light chains referred to as L-chains.

As used herein and in the claims, the phrase “an immunological portion of an antibody” includes a Fab fragment of an antibody, a Fv fragment of an antibody, a heavy chain of an antibody, a light chain of an antibody, a heterodimer consisting of a heavy chain and a light chain of an antibody, a variable fragment of a light chain of an antibody, a variable fragment of a heavy chain of an antibody, and a single chain variant of an antibody, which is also known as scFv. In addition, the term includes chimeric immunoglobulins which are the expression products of fused genes derived from different species, one of the species can be a human, in which case a chimeric immunoglobulin is said to be humanized. Typically, an immunological portion of an antibody competes with the intact antibody from which it was derived for specific binding to an antigen.

Optionally, an antibody or preferably an immunological portion of an antibody, can be chemically conjugated to, or expressed as, a fusion protein with other proteins. For purposes of this specification and the accompanying claims, all such fused proteins are included in the definition of antibodies or an immunological portion of an antibody.

As used herein the terms “immunogenic agent” or “immunogen” or “antigen” are used interchangeably to describe a molecule capable of inducing an immunological response against itself on administration to a recipient, either alone, in conjunction with an adjuvant, or presented on a display vehicle.

D. Treatment Methods

A method of the present invention includes treatment for a disease or condition caused by a staphylococcus pathogen. A bacterium or vaccine of the present invention can be administered to induce an immune response in a person infected with staphylococcus, suspected of having been exposed to staphylococcus, or at risk of such exposure. Methods may be employed with respect to individuals who have tested positive for exposure to staphylococcus or who are deemed to be at risk for infection based on possible exposure.

In particular, the invention encompasses a method of treatment for staphylococcal infection, particularly hospital acquired nosocomial infections. The bacteria and vaccines of the invention are particularly advantageous to use in cases of elective surgery. Such patients will know the date of surgery in advance and could be inoculated in advance. The bacteria and vaccines of the invention are also advantageous to use to inoculate health care workers.

In some embodiments, the treatment is administered in the presence of biological response modifiers. Furthermore, in some examples, treatment comprises administration of other agents commonly used against bacterial infection, such as one or more antibiotics.

The use of vaccines, discussed below, to treat or prevent infections (active immunization) is specifically contemplated, as is the transfer of immune effectors from a vaccinated patient to another subject (passive immunization).

E. Combination Therapy

The compositions and related methods of the present invention, particularly administration of a bacterium or vaccine, may also be used in combination with the administration of traditional therapies. These include, but are not limited to, the administration of antibiotics such as streptomycin, ciprofloxacin, doxycycline, gentamycin, chloramphenicol, trimethoprim, sulfamethoxazole, ampicillin, tetracycline or various combinations of antibiotics.

In one aspect, it is contemplated that a vaccine and/or therapy is used in conjunction with antibacterial treatment. Alternatively, the vaccine therapy may precede or follow the other agent treatment by intervals ranging from minutes to weeks. In embodiments where the other agents and/or vaccine are administered separately, one would generally ensure that a significant period of time did not expire between the time of each delivery, such that the agent and vaccine composition would still be able to exert an advantageously combined effect on the subject. In such instances, it is contemplated that one may administer both modalities within about 12-24 h of each other or within about 6-12 h of each other. In some situations, it may be desirable to extend the time period for administration significantly, where several days (2, 3, 4, 5, 6 or 7) to several weeks (1, 2, 3, 4, 5, 6, 7 or 8) lapse between the respective administrations.

Various combinations may be employed, for example, where the vaccine therapy is “A” and the other therapy is “B”:

| | | | | | | | |
|---|---|---|---|---|---|---|---|
| A/B/A | B/A/B | B/B/A | A/A/B | A/B/B | B/A/A | A/B/B/B | B/A/B/B |
| B/B/B/A | B/B/A/B | A/A/B/B | A/B/A/B | A/B/B/A | B/B/A/A | | |
| B/A/B/A | B/A/A/B | A/A/A/B | B/A/A/A | A/B/A/A | A/A/B/A | | |

Administration of the immunogenic compositions of the present invention to a patient/subject will follow general protocols for the administration of such compounds, taking into account the toxicity, if any, of the vaccine or other compositions described herein. It is expected that the treatment cycles would be repeated as necessary. It also is contemplated that various standard therapies, such as hydration, may be applied in combination with the described therapy. Secondary agents include antibiotics and polyclonal antisera (WO00/15238, WO00/12132) or monoclonal antibodies against lipoteichoic acid (WO98/57994).

### V. Vaccines and Other Pharmaceutical Compositions and Administration

A. Vaccines

The present invention includes methods for preventing or ameliorating staphylococcal infections, particularly hospital acquired nosocomial infections. As such, the invention contemplates vaccines for use in both active and passive immunization embodiments. The bacteria and vaccines are described elsewhere in this document

The preparation of vaccines is generally well understood in the art, as exemplified by U.S. Pat. Nos. 4,608,251; 4,601,903; 4,599,231; 4,599,230; 4,596,792; and 4,578,770, all of which are incorporated herein by reference. Typically, such vaccines are prepared as injectables either as liquid solutions or suspensions: solid forms suitable for solution in or suspension in liquid prior to injection may also be prepared. The preparation may also be emulsified. The active immunogenic ingredient is often mixed with excipients that are pharmaceutically acceptable and compatible with the active ingredient. Suitable excipients are, for example, water, saline, dextrose, glycerol, ethanol, or the like and combinations thereof. In addition, if desired, the vaccine may contain amounts of auxiliary substances such as wetting or emulsifying agents, pH buffering agents, or adjuvants that enhance the effectiveness of the vaccines. In specific embodiments, vaccines are formulated with a combination of substances, as described in U.S. Pat. Nos. 6,793,923 and 6,733,754, which are incorporated herein by reference.

Vaccines may be conventionally administered parenterally, by injection, for example, either subcutaneously or intramuscularly. Additional formulations which are suitable for other modes of administration include suppositories and, in some cases, oral formulations. For suppositories, traditional binders and carriers may include, for example, polyalkalene glycols or triglycerides: such suppositories may be formed from mixtures containing the active ingredient in the range of about 0.5% to about 10%, preferably about 1% to about 2%. Oral formulations include such normally employed excipients as, for example, pharmaceutical grades of mannitol, lactose, starch, magnesium stearate, sodium saccharine, cellulose, magnesium carbonate and the like. These compositions take the form of solutions, suspensions, tablets, pills, capsules, sustained release formulations or powders and contain about 10% to about 95% of active ingredient, preferably about 25% to about 70%.

Typically, vaccines are administered in a manner compatible with the dosage formulation, and in such amount as will be therapeutically effective and immunogenic. The quantity to be administered depends on the subject to be treated, including the capacity of the individual's immune system to synthesize antibodies and the degree of protection desired. Precise amounts of active ingredient required to be administered depend on the judgment of the practitioner. However, suitable dosage ranges are of the order of several hundred micrograms of active ingredient per vaccination. Suitable regimes for initial administration and booster shots are also variable, but are typified by an initial administration followed by subsequent inoculations or other administrations.

The manner of application may be varied widely. Any of the conventional methods for administration of a vaccine are applicable. These are believed to include oral application within a solid physiologically acceptable base or in a physiologically acceptable dispersion, parenterally, by injection and the like. The dosage of the vaccine will depend on the route of administration and will vary according to the size and health of the subject.

In certain instances, it will be desirable to have multiple administrations of the vaccine, e.g., 2, 3, 4, 5, 6 or more administrations. The vaccinations can be at 1, 2, 3, 4, 5, 6, 7, 8, to 5, 6, 7, 8, 9, 10, 11, 12 twelve week intervals, including all ranges there between. Periodic boosters at intervals of 1-5 years will be desirable to maintain protective levels of the antibodies. The course of the immunization may be followed by assays for antibodies against the antigens, as described in U.S. Pat. Nos. 3,791,932; 4,174,384 and 3,949,064.

The immunogenicity of polypeptide or peptide compositions can be enhanced by the use of non-specific stimulators of the immune response, known as biological response modifiers. Such agents include all acceptable immunostimulatory compounds, such as cytokines, toxins, or synthetic compositions, including adjuvants that can (1) trap the antigen in the body to cause a slow release; (2) attract cells involved in the immune response to the site of administration; (3) induce proliferation or activation of immune system cells; or (4) improve the spread of the antigen throughout the subject's body.

Biological response modifiers include, but are not limited to, oil-in-water emulsions, water-in-oil emulsions, mineral salts, polynucleotides, and natural substances, and specific examples that may be used include IL-1, IL-2, IL-4, IL-7, IL-12, γ-interferon, GMCSP, BCG, aluminum salts, such as aluminum hydroxide or other aluminum compound, MDP compounds, such as thur-MDP and nor-MDP, CGP (MTP-PE), lipid A, and monophosphoryl lipid A (MPL). RIBI, which contains three components extracted from bacteria, MPL, trehalose dimycolate (TDM), and cell wall skeleton (CWS) in a 2% squalene/Tween 80 emulsion. MHC antigens may even be used. Others agents or methods are exemplified in U.S. Pat. Nos. 6,814,971, 5,084,269, 6,656,462, each of which is incorporated herein by reference).

Various methods of achieving adjuvant affect for the vaccine includes use of agents such as aluminum hydroxide or phosphate (alum), commonly used as about 0.05 to about 0.1% solution in phosphate buffered saline, admixture with synthetic polymers of sugars (Carbopol®) used as an about 0.25% solution, aggregation of the protein in the vaccine by heat treatment with temperatures ranging between about 70° to about 101° C. for a 30-second to 2-minute period, respectively. Aggregation by reactivating with pepsin-treated (Fab) antibodies to albumin; mixture with bacterial cells (e.g., C. parvum), endotoxins or lipopolysaccharide components of Gram-negative bacteria; emulsion in physiologically acceptable oil vehicles (e.g., mannide mono-oleate (Aracel A)); or emulsion with a 20% solution of a perfluorocarbon (Fluosol-DA®) used as a block substitute may also be employed to produce an adjuvant effect.

Examples of and often preferred adjuvants include complete Freund's adjuvant (a non-specific stimulator of the immune response containing killed Mycobacterium tuberculosis), incomplete Freund's adjuvants, and aluminum hydroxide.

In some aspects, it is preferred that the agent be selected to be a preferential inducer of either a Th1 or a Th2 type of response. High levels of Th1-type cytokines tend to favor the induction of cell mediated immune responses to a given antigen, while high levels of Th2-type cytokines tend to favor the induction of humoral immune responses to the antigen.

The distinction of Th1 and Th2-type immune response is not absolute. In reality an individual will support an immune response which is described as being predominantly Th1 or predominantly Th2. However, it is often convenient to consider the families of cytokines in terms of that described in murine CD4+ T cell clones by Mosmann and Coffman (Mosmann, and Coffman, 1989). Traditionally, Th1-type responses are associated with the production of the INF-γ and IL-2 cytokines by T-lymphocytes. Other cytokines often directly associated with the induction of Th1-type immune responses are not produced by T-cells, such as IL-12. In contrast, Th2-type responses are associated with the secretion of IL-4, IL-5, IL-6, IL-10.

Other than traditional adjuvants, biologic response modifiers (BRM) include agents shown to upregulate T cell immunity or downregulate suppresser cell activity. Such BRMs include, but are not limited to, Cimetidine (CIM; 1200 mg/d) (Smith/Kline, PA); or low-dose Cyclophosphamide (CYP; 300 mg/m²) (Johnson/Mead, NJ) and cytokines such as γ-interferon, IL-2, or IL-12 or genes encoding proteins involved in immune helper functions, such as B-7.

B. General Pharmaceutical Compositions

In some embodiments, pharmaceutical compositions are administered to a subject. Different aspects of the present invention involve administering an effective amount of a composition to a subject. Additionally, such compounds can be administered in combination with an antibiotic or an antibacterial. Such compositions will generally be dissolved or dispersed in a pharmaceutically acceptable carrier or aqueous medium.

In addition to the compounds formulated for parenteral administration, such as those for intravenous or intramuscular injection, other pharmaceutically acceptable forms include, e.g., tablets or other solids for oral administration; time release capsules; and any other form currently used, including creams, lotions, mouthwashes, inhalants and the like.

The active compounds of the present invention can be formulated for parenteral administration, e.g., formulated for injection via the intravenous, intramuscular, sub-cutaneous, or even intraperitoneal routes. The preparation of an aqueous composition that contains a compound or compounds that increase the expression of an MHC class I molecule will be known to those of skill in the art in light of the present disclosure. Typically, such compositions can be prepared as injectables, either as liquid solutions or suspensions; solid forms suitable for use to prepare solutions or suspensions upon the addition of a liquid prior to injection can also be prepared; and, the preparations can also be emulsified.

Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions; formulations including sesame oil, peanut oil, or aqueous propylene glycol; and sterile powders for the extemporaneous preparation of sterile injectable solutions or dispersions. In all cases the faun must be sterile and must be fluid to the extent that it may be easily injected. It also should be stable under the conditions of manufacture and storage and must be preserved against the contaminating action of microorganisms, such as bacteria and fungi.

The proteinaceous compositions may be formulated into a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier also can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

Administration of the compositions according to the present invention will typically be via any common route. This includes, but is not limited to oral, nasal, or buccal administration. Alternatively, administration may be by orthotopic, intradermal, subcutaneous, intramuscular, intraperitoneal, intranasal, or intravenous injection. In certain embodiments, a vaccine composition may be inhaled (e.g., U.S. Pat. No. 6,651,655, which is specifically incorporated by reference). Such compositions would normally be administered as pharmaceutically acceptable compositions that include physiologically acceptable carriers, buffers or other excipients. As used herein, the term “pharmaceutically acceptable” refers to those compounds, materials, compositions, and/or dosage forms which are, within the scope of sound medical judgment, suitable for contact with the tissues of human beings and animals without excessive toxicity, irritation, allergic response, or other problem complications commensurate with a reasonable benefit/risk ratio. The term “pharmaceutically acceptable carrier,” means a pharmaceutically acceptable material, composition or vehicle, such as a liquid or solid filler, diluent, excipient, solvent or encapsulating material, involved in carrying or transporting a chemical agent.

For parenteral administration in an aqueous solution, for example, the solution should be suitably buffered, if necessary, and the liquid diluent first rendered isotonic with sufficient saline or glucose. These particular aqueous solutions are especially suitable for intravenous, intramuscular, subcutaneous, and intraperitoneal administration. In this connection, sterile aqueous media which can be employed will be known to those of skill in the art in light of the present disclosure. For example, one dosage could be dissolved in isotonic NaCl solution and either added to hypodermoclysis fluid or injected at the proposed site of infusion, (see for example, Remington's Pharmaceutical Sciences, 1990). Some variation in dosage will necessarily occur depending on the condition of the subject. The person responsible for administration will, in any event, determine the appropriate dose for the individual subject.

An effective amount of therapeutic or prophylactic composition is determined based on the intended goal. The term “unit dose” or “dosage” refers to physically discrete units suitable for use in a subject, each unit containing a predetermined quantity of the composition calculated to produce the desired responses discussed above in association with its administration, i.e., the appropriate route and regimen. The quantity to be administered, both according to number of treatments and unit dose, depends on the protection desired.

Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition.

Upon formulation, solutions will be administered in a manner compatible with the dosage formulation and in such amount as is therapeutically or prophylactically effective. The formulations are easily administered in a variety of dosage forms, such as the type of injectable solutions described above.

C. Antibodies and Passive Immunization

Another aspect of the invention is a method of preparing an immunoglobulin or serum for use in prevention or treatment of staphylococcal infection comprising the steps of immunizing a recipient or donor with the vaccine of the invention and isolating immunoglobulin from the recipient or donor. An immunoglobulin or serum prepared by this method is a further aspect of the invention. A pharmaceutical composition comprising the immunoglobulin of the invention and a pharmaceutically acceptable carrier is a further aspect of the invention which could be used in the manufacture of a medicament for the treatment or prevention of staphylococcal disease. A method for treatment or prevention of staphylococcal infection comprising a step of administering to a patient an effective amount of the pharmaceutical preparation of the invention is a further aspect of the invention.

Inocula for polyclonal antibody production are typically prepared by dispersing the antigenic composition in a physiologically tolerable diluent such as saline or other adjuvants suitable for human use to form an aqueous composition. An immunostimulatory amount of inoculum is administered to a mammal and the inoculated mammal is then maintained for a time sufficient for the antigenic composition to induce protective antibodies.

The antibodies can be isolated to the extent desired by well known techniques such as affinity chromatography (Harlow and Lane, 1988). Antibodies can include antiserum preparations from a variety of commonly used animals, e.g. goats, primates, donkeys, swine, horses, guinea pigs, rats or man.

An immunoglobulin produced in accordance with the present invention can include whole antibodies, antibody fragments or subfragments. Antibodies can be whole immunoglobulins of any class (e.g., IgG, IgM, IgA, IgD or IgE), chimeric antibodies or hybrid antibodies with dual specificity to two or more antigens of the invention. They may also be fragments (e.g., F(ab′)2, Fab′, Fab, Fv and the like) including hybrid fragments. An immunoglobulin also includes natural, synthetic, or genetically engineered proteins that act like an antibody by binding to specific antigens to form a complex.

A vaccine of the present invention can be administered to a recipient who then acts as a source of immunoglobulin, produced in response to challenge from the specific vaccine. A subject thus treated would donate plasma from which hyperimmune globulin would be obtained via conventional plasma fractionation methodology. The hyperimmune globulin would be administered to another subject in order to impart resistance against or treat staphylococcal infection. Hyperimmune globulins of the invention are particularly useful for treatment or prevention of staphylococcal disease in infants, immune compromised individuals, or where treatment is required and there is no time for the individual to produce antibodies in response to vaccination.

An additional aspect of the invention is a pharmaceutical composition comprising two of more monoclonal antibodies (or fragments thereof; preferably human or humanised) reactive against at least two constituents of the immunogenic composition of the invention, which could be used to treat or prevent infection by Gram positive bacteria, preferably staphylococci, more preferably S. aureus or S. epidermidis. Such pharmaceutical compositions comprise monoclonal antibodies that can be whole immunoglobulins of any class, chimeric antibodies, or hybrid antibodies with specificity to two or more antigens of the invention. They may also be fragments (e.g., F(ab′)2, Fab′, Fab, Fv and the like) including hybrid fragments.

Methods of making monoclonal antibodies are well known in the art and can include the fusion of splenocytes with myeloma cells (Kohler and Milstein, 1975; Harlow and Lane, 1988). Alternatively, monoclonal Fv fragments can be obtained by screening a suitable phage display library (Vaughan et al., 1998). Monoclonal antibodies may be humanized or part humanized by known methods.

### VI. Examples

The following examples are given for the purpose of illustrating various embodiments of the invention and are not meant to limit the present invention in any fashion. One skilled in the art will appreciate readily that the present invention is well adapted to carry out the objects and obtain the ends and advantages mentioned, as well as those objects, ends and advantages inherent herein. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1

### Materials & Methods

Bacterial Strains and Culturing Conditions. Staphylococci were cultured with tryptic soy broth (TSB) or agar at 37° C. E. coli strains DH5a and BL21(DE3) were cultured with Luria borth (LB) or agar at 37° C. Ampicillin (100 μg ml⁻¹ for pET15b), erythromycin (200 μg ml⁻¹ for bursa aurealis variants) and spectinomycin (200 μg ml⁻¹ for the spa deletion variant) were used for the selection of antibiotic resistance traits.

Mutagenesis. Insertional mutations from the Phoenix library were transduced into S. aureus Newman (Bae et al., 2004; Cheng et al., 2010). Each mutant carries the transposon bursa aurealis containing an erythromycin resistance cassette in the gene of interest, and mutations were verified as described previously (Bae et al., 2004). The spa gene on the chromosome of S. aureus Newman were deleted by allelic replacement as described previously (Kim et al., 2010; Bae & Schneewind, 2005).

Cloning and Purification. Coding sequences for ClfA, SdrD, and FnBPB were PCR amplified using S. aureus Newman template DNA (Stranger-Jones et al., 2006). PCR products were cloned into pET15b to express recombinant proteins with N-terminal His₆-tag fusion. Cloning of non-toxigenic protein A was described previously (Kim et al., 2010). Plasmids were transformed into BL21(DE3). Overnight cultures of transformants were diluted 1:100 into fresh media and grown at 37° C. to an OD₆₀₀ 0.5, at which point cultures were induced with 1 mM isopropyl β-D-1-thiogalatopyranoside (IPTG) and grown for an additional three hours. Bacterial cells were sedimented by centrifugation, suspended in column buffer (50 mM Tris-HCl, pH 7.5, 150 mM NaCl) and disrupted with a French pressure cell at 14,000 psi. Lysates were cleared of membrane and insoluble components by ultracentrifugation at 40,000×g. Proteins in the soluble lysate were subjected to nickel-nitrilotriacetic acid (Ni-NTA, Qiagen) affinity chromatography. Proteins were eluted in column buffer containing successively higher concentrations of imidazole (100-500 mM). Protein concentrations were determined by bicinchonic acid (BCA) assay (Thermo Scientific).

Live-attenuated Vaccine and Renal Abscess Model. Overnight cultures of S. aureus Newman and its isogenic mutants were diluted 1:100 into fresh TSB and grown for 2 hours at 37° C. Staphylococci were sedimented, washed and suspended PBS at OD₆₀₀ of 0.4 (˜1×10⁸ CFU ml⁻¹). Inocula were quantified by spreading sample aliquots on TSA and enumerating colonies formed. BALB/c mice (4 week-old, female, Charles River Laboratories) were anesthetized via intraperitoneal injection with 100 mg ml⁻¹ ketamine and 20 mg ml⁻¹ xylazine per kilogram of body weight. Mice were infected with 100 μl of bacterial suspension (1×10⁷ CFU) by retro-orbital injection. On day 19 following infection, cohorts of mice were treated with antibiotics, a mixture of ampicillin (1 mg ml⁻¹) and chloramphenicol (1 mg ml⁻¹) in water for 3 days, followed by next 4 days with clean water. On day 26, mice were challenged with 100 μl of S. aureus Newman (1×10⁷ CFU) by retro-orbital injection. Cohorts of mice were killed by CO₂ inhalation on day 18 and 30 post initial infection. Both kidneys were removed, and the staphylococcal load in right kidney was analyzed by homogenizing renal tissue with PBS, 0.1% Triton X-100. Serial dilutions of homogenate were spread on TSA or TSA containing antibiotics (Erm or Spec) and incubated for colony formation. The left kidney was examined by histopathology. Briefly, kidneys were fixed in 10% formalin for 24 hours at room temperature. Tissues were embedded in paraffin, thin-sectioned, stained with hematoxylin-eosin, and inspected by light microscopy to enumerate abscess lesions. Also, hyper-immune sera were collected via cardiac puncture and analyzed against components of the staphylococcal antigen matrix. All mouse experiments were performed in accordance with the institutional guidelines following experimental protocol review and approval by the Institutional Biosafety Committee (IBC) and the Institutional Animal Care and Use Committee (IACUC) at the University of Chicago.

Active Immunization. BALB/c mice (3 week-old, female, Charles River Laboratories) were immunized with 25 μg protein emulsified in Complete Freund's Adjuvant (Difco) by intramuscular injection. For booster immunizations, proteins were emulsified in Incomplete Freund's Adjuvant and injected 11 days following the initial immunization. On day 20 following immunization, 5 mice were bled to obtain sera for specific antibody titers by enzyme-linked immunosorbent assay (ELISA). On day 21, all mice were challenged with 1×10⁷ CFU S. aureus Newman. Four and eighteen days following challenge, kidneys were removed during necropsy, and renal tissue was analyzed for staphylococcal load or histopathology. Also, hyper-immune sera were collected via cardiac puncture and analyzed against components of the staphylococcal antigen matrix.

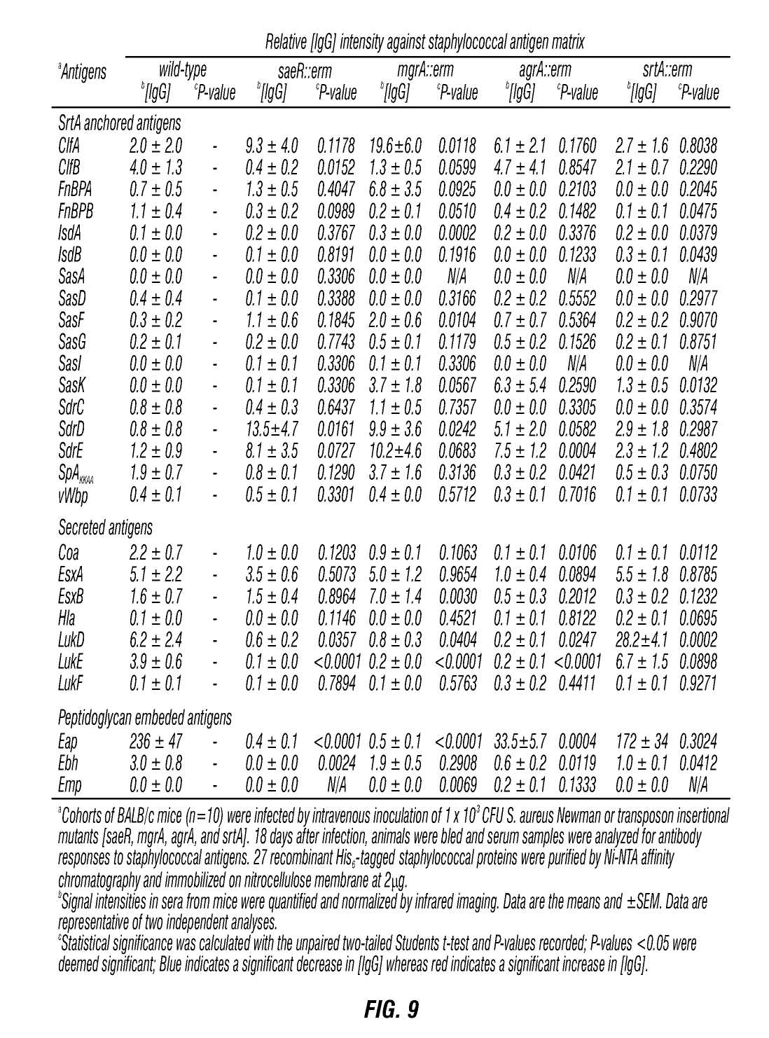

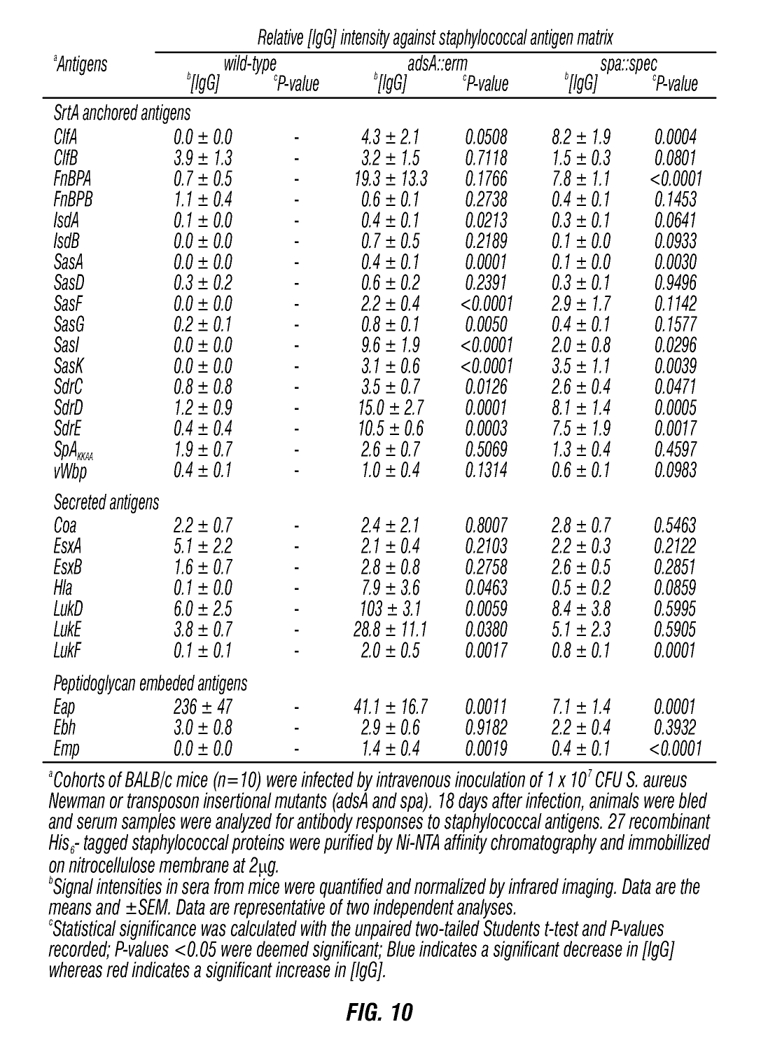

Antibody Quantification. For the staphylococcal antigen matrix, nitrocellulose membrane was blotted with 2 μg of a collection of Ni-NTA affinity purified recombinant His₆ tagged staphylococcal proteins (Kim et al., 2010). Signal intensities in mouse sera were quantified and normalized using anti-His₆ antibody with the Odyssey™ (FIGS. 9-11).

Statistical Analysis. Unpaired two-tailed Student's t tests were performed to analyze the statistical significance. Linear regression analysis was performed using Graphpad Prism.

### Example 2

### Results

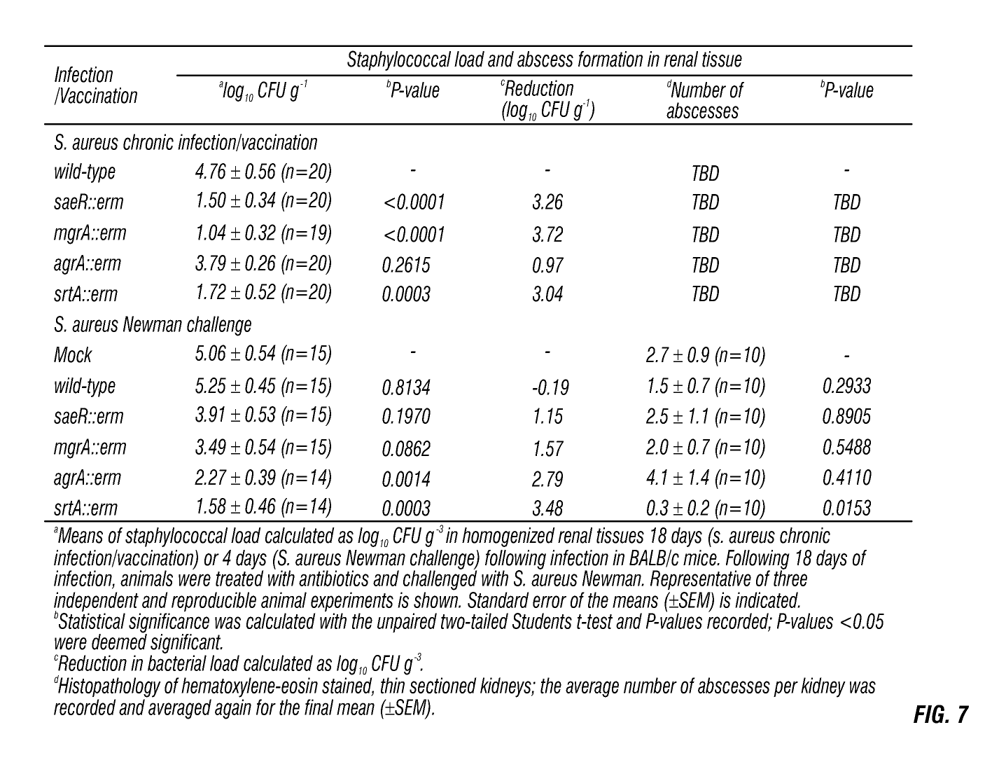

Four weeks old BALB/c mice were infected by intravenous inoculation with S. aureus Newman (wild-type), or saeR, mgrA, agrA, or srtA transposon insertional mutants. Eighteen days later, bacterial loads in kidney tissues were examined. saeR and mgrA showed substantially reduced infection, srtA showed intermediate infection, and agrA showed infection approaching that of wild-type (FIGS. 1A-K and FIG. 7).

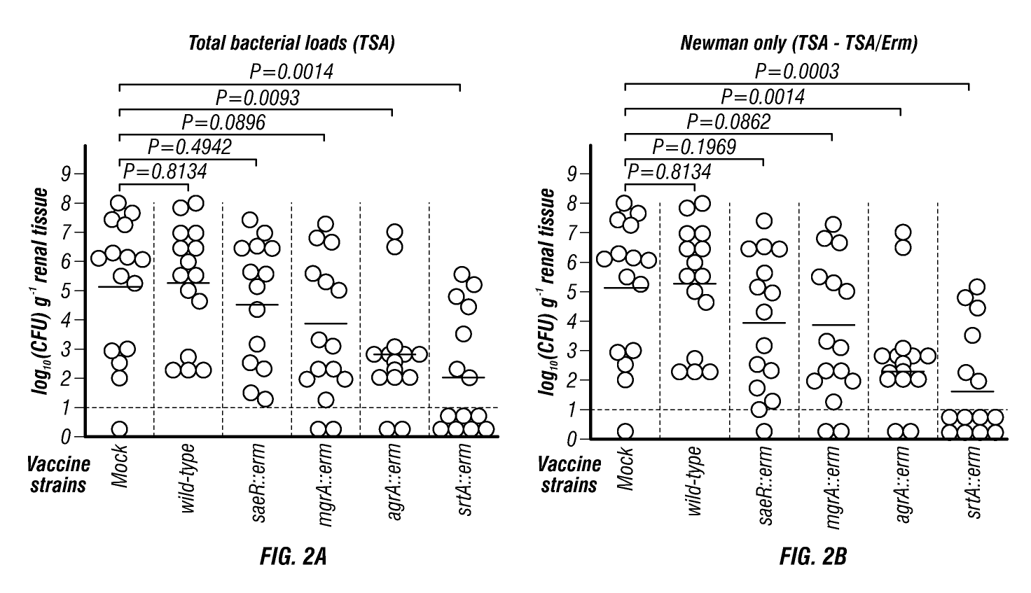

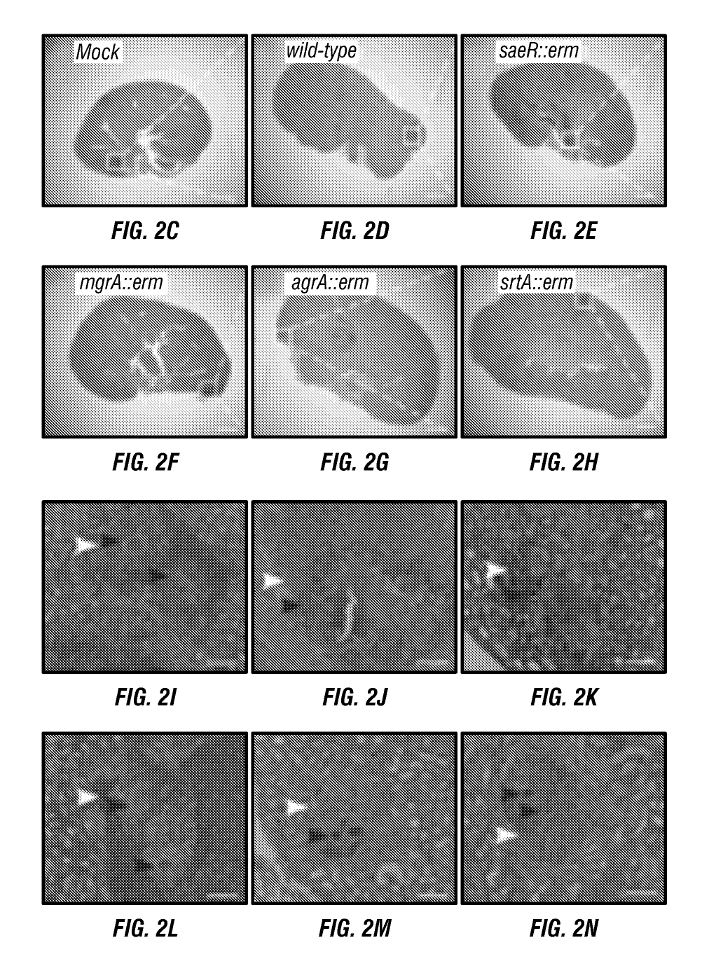

Four weeks old BALB/c mice were infected by intravenous inoculation either with PBS (mock), S. aureus Newman (wild-type), or saeR, mgrA, agrA, or srtA transposon insertional mutants. Following antibiotic treatment, mice were challenged by intravenous inoculation with S. aureus Newman (wild-type). Four days later, total bacterial load (TSA) in renal tissues and Staphylococcal burden of the remaining wild-type S. aureus Newman (TSA-TSA/Erm) in renal tissues was examined. saeR and mgrA showed infection substantially similar to wild-type, while srtA showed substantial reduction as did agrA, but the latter was slightly less than the former (FIGS. 2A-N and FIG. 7).

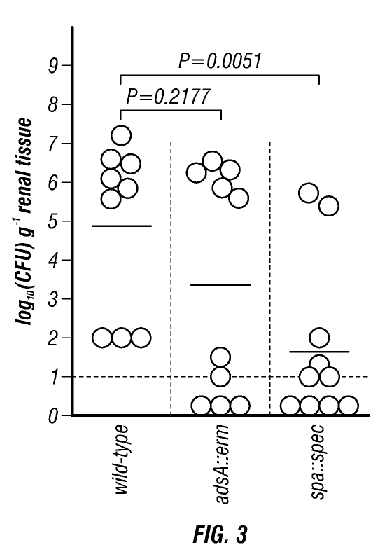

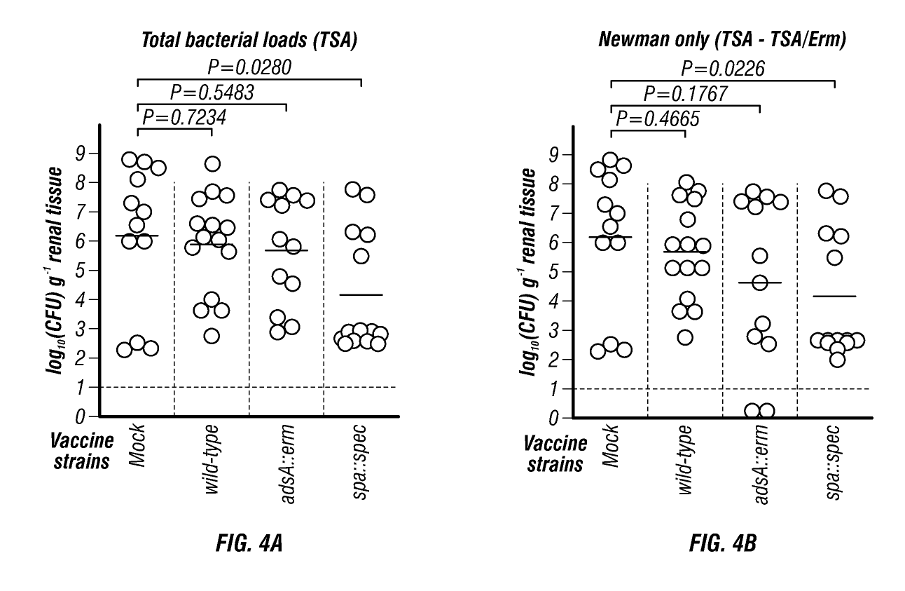

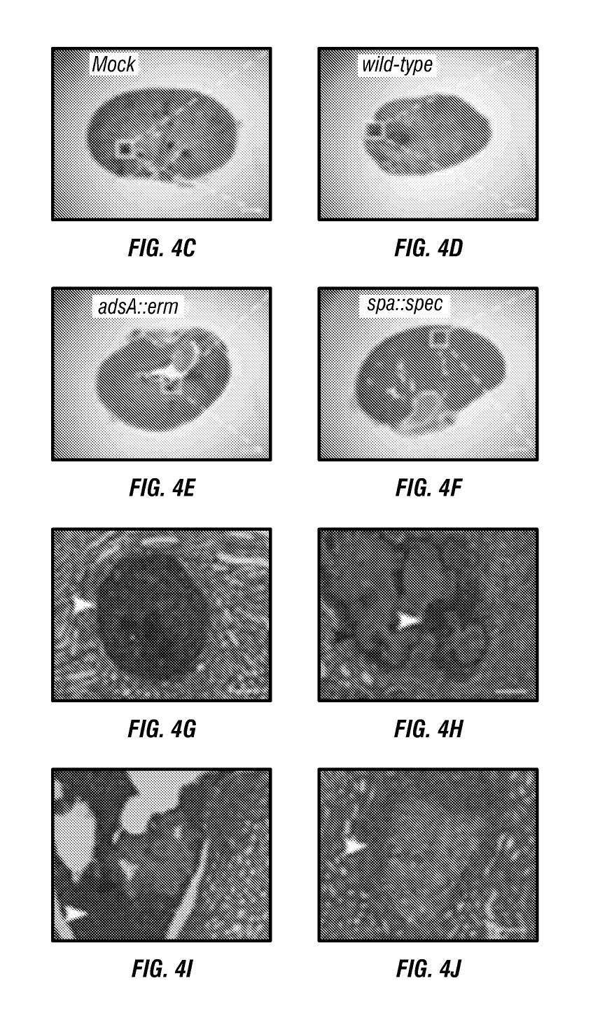

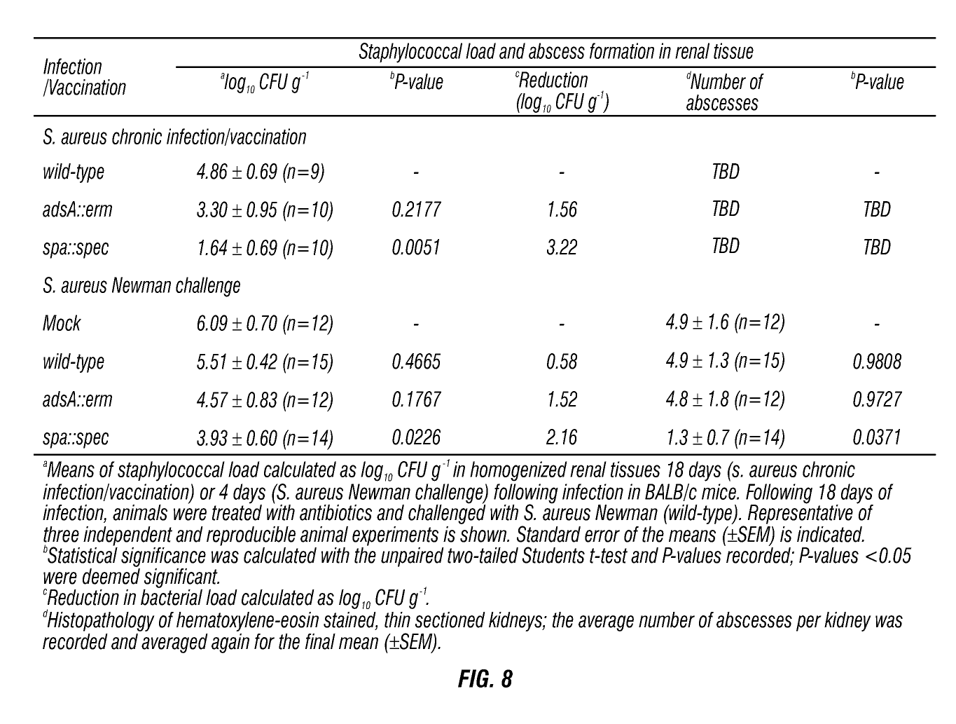

Four weeks old BALB/c mice were infected by intravenous inoculation with S. aureus Newman (wild-type), adsA or spa transposon insertional mutants. 18 days later, bacterial loads in kidney tissues were examined. adsA mutant had slightly reduced infection, while spa mutant had substantially reduced infection (FIG. 3 and FIG. 8).

Four weeks old BALB/c mice were infected by intravenous inoculation either with PBS (mock) or S. aureus Newman (wild-type), adsA, or spa transposon insertional mutants. Following antibiotic treatment, mice were challenged by intravenous inoculation with S. aureus Newman (wild-type). Four days later, total bacterial loads (TSA) in renal tissues and remaining burden of S. aureus Newman (TSA-TSA/Erm) in renal tissues was examined. While mock, S. aureus Newman (wild-type), and adsA were all quite similar, the spa transposon mutant showed lower TSA. It also showed the lowest TSA-TSA/Erm, although the adsA mutant performed somewhat better in that assay as compared to the TSA assay (FIG. 8).

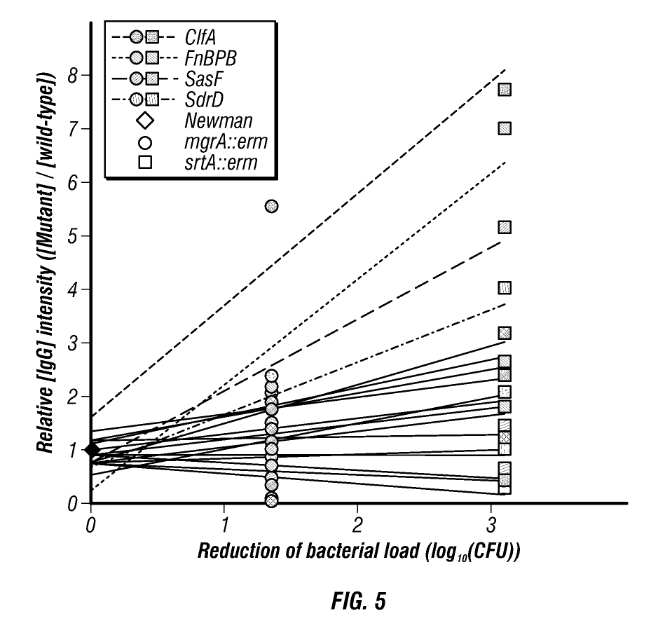

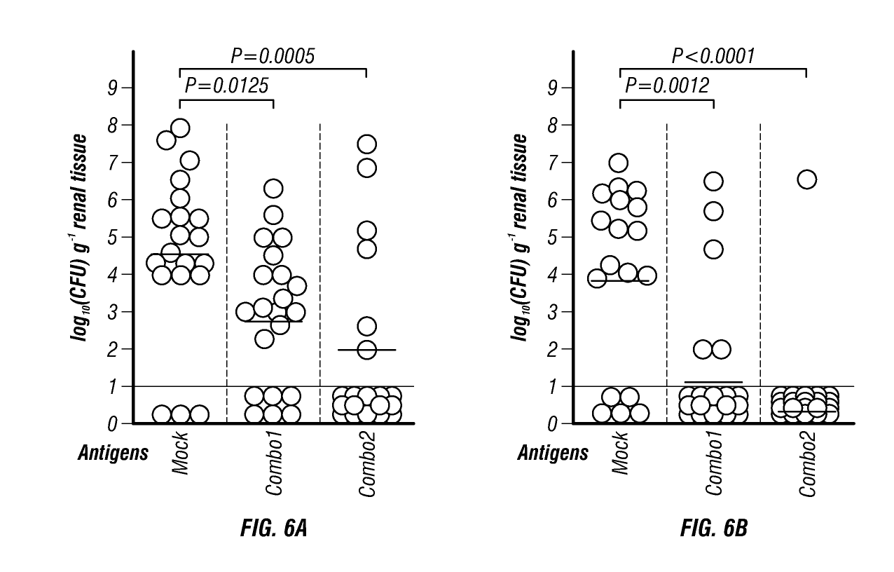

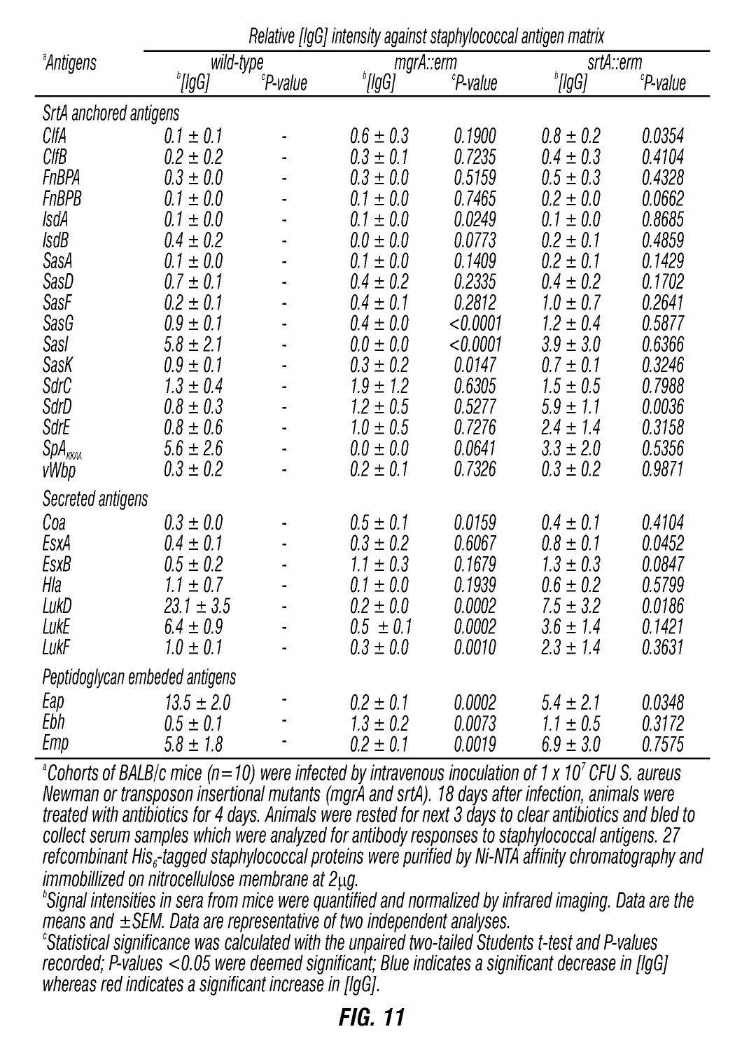

BALB/c mice were infected with S. aureus Newman, mgrA or srtA mutant strains for 18 days, and infection was cleared with chloramphenicol and ampicillin treatment. Cohorts of animals were terminally bled and immune sera samples were collected and analyzed against the components of the staphylococcal antigen matrix (FIGS. 9, 11). Another cohort of animals was then challenged with S. aureus Newman and bacterial load (log₁₀(CFU)) in kidney tissue homogenate was analyzed after necropsy on day 4. Correlations between bacterial load reduction (mutant strains compare to wild-type) and humoral immune responses toward 27 staphylococcal antigens ([mutant IgG]/[wild-type IgG]) were plotted and are shown in FIG. 5.

BALB/c mice were either mock immunized with PBS/adjuvant or injected with 25 μg of each antigen (Combo 1, ClfA+SdrD+FnBPB; Combo 2, Combo 1+SpAKKAA). Immunized mice were challenged by intravenous inoculation with S. aureus Newman. Bacterial loads in kidney tissues were examined at day 4 and day 18 post-challenge. The Combo 2 provided the greatest reduction in infection, although Combo 1 also was effective.

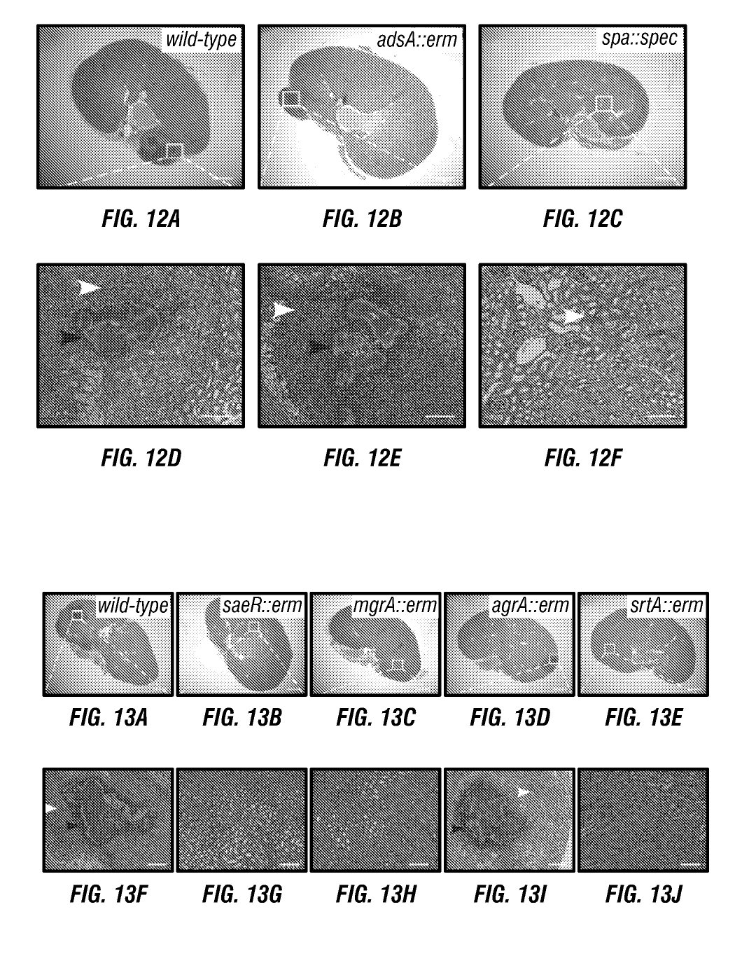

Protein A is Required for Staphylococcal Persistence in Host Tissues. Previous work implemented staphylococcal protein A (SpA), a B cell superantigen that binds to the Fc-γ and Fab VH3 portions of immunoglobulins, as a factor in the pathogen's strategy to modulate host adaptive immune responses. mgrA and srtA variants harbor the wild-type spa gene, however srtA, not mgrA, is required for the cell wall anchoring and surface display of protein A. Adenosine synthase A (AdsA), an enzyme that cleaves nucleotides to trigger the build-up of adenosine in infected tissues, is also anchored by SrtA. AdsA activity is thought to signal via adenosine receptors on the surface of lymphocytes as well as cells of the myeloid lineage, interfering with host innate and adaptive immune responses. The inventors contemplated that the inability of srtA mutants to display wild-type levels of SpA or AdsA is associated with the attribute of eliciting protective immunity. Eighteen days after inoculation of mice, the spa mutant displayed a decrease in bacterial load and abscess lesions (FIG. 12). The adsA mutant also displayed a reduction in bacterial load, however this defect was not significantly different from the wild-type parent (FIG. 12). Thus, even though adsA mutants are defective for the establishment of abscess lesion on day five following injection, the variants can persist in infected host tissues (FIG. 12).

Sortase A Mutants Elicit Protective Immune Responses in Mice. Following injection into the bloodstream of mice, S. aureus seeds abscesses in all organ systems. Within four days, staphylococci grow at the center of these lesion surrounded by a pseudocapsule of fibrin deposits as well as layers of live and dead immune cells (FIG. 13). Abscesses migrate to the surface of organs where they rupture and release pathogens into body fluids with ensuing establishment of new lesions. Even after 18-30 days, infected hosts mount humoral immune responses against only few staphylococcal antigens. Animals with a history of staphylococcal infection do not acquire protective immunity. For example, following treatment of staphylococcal infection with chloramphenicol, mice are again challenged with the same S. aureus strain. The subsequent challenge produces similar pathology, bacterial burden and persistence as occurs with naïve mice. These observations are in agreement with a model whereby S. aureus infection actively suppresses the development of adaptive immune responses. To identify staphylococcal mutants unable to suppress host immune responses, the inventors analyzed variants lacking key regulatory genes. AgrA is the response regulator of the two-component sensory transduction module (AgrCA) responding to AgrBD-derived quorum signals; it regulates the expression of exoprotein and surface protein genes. MgrA is the cytoplasmic sensor of oxidative stress, as occurs when S. aureus is phagocytosed by immune cells. Another two-component regulatory system, SaeRS, controls key virulence factors secreted by S. aureus. Mutants lacking saeR or mgrA displayed severe virulence defects, allowing infected mice to clear these infections

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 3,791,932
U.S. Pat. No. 3,949,064
U.S. Pat. No. 4,174,384
U.S. Pat. No. 4,338,298
U.S. Pat. No. 4,356,170
U.S. Pat. No. 4,367,110
U.S. Pat. No. 4,372,945
U.S. Pat. No. 4,452,901
U.S. Pat. No. 4,474,757
U.S. Pat. No. 4,554,101
U.S. Pat. No. 4,578,770
U.S. Pat. No. 4,596,792
U.S. Pat. No. 4,599,230
U.S. Pat. No. 4,599,231
U.S. Pat. No. 4,601,903
U.S. Pat. No. 4,608,251
U.S. Pat. No. 4,683,195
U.S. Pat. No. 4,683,202
U.S. Pat. No. 4,684,611
U.S. Pat. No. 4,690,915
U.S. Pat. No. 4,690,915
U.S. Pat. No. 4,748,018
U.S. Pat. No. 4,800,159
U.S. Pat. No. 4,879,236
U.S. Pat. No. 4,952,500
U.S. Pat. No. 5,084,269
U.S. Pat. No. 5,199,942
U.S. Pat. No. 5,221,605
U.S. Pat. No. 5,238,808
U.S. Pat. No. 5,302,523
U.S. Pat. No. 5,310,687
U.S. Pat. No. 5,322,783
U.S. Pat. No. 5,384,253
U.S. Pat. No. 5,464,765
U.S. Pat. No. 5,512,282
U.S. Pat. No. 5,512,282
U.S. Pat. No. 5,538,877
U.S. Pat. No. 5,538,880
U.S. Pat. No. 5,548,066
U.S. Pat. No. 5,550,318
U.S. Pat. No. 5,563,055
U.S. Pat. No. 5,580,859
U.S. Pat. No. 5,589,466
U.S. Pat. No. 5,591,616
U.S. Pat. No. 5,610,042
U.S. Pat. No. 5,620,896
U.S. Pat. No. 5,648,240
U.S. Pat. No. 5,656,610
U.S. Pat. No. 5,702,932
U.S. Pat. No. 5,736,524
U.S. Pat. No. 5,780,448
U.S. Pat. No. 5,789,215
U.S. Pat. No. 5,801,234
U.S. Pat. No. 5,840,846
U.S. Pat. No. 5,843,650
U.S. Pat. No. 5,846,709
U.S. Pat. No. 5,846,783
U.S. Pat. No. 5,849,497
U.S. Pat. No. 5,849,546
U.S. Pat. No. 5,849,547
U.S. Pat. No. 5,858,652
U.S. Pat. No. 5,866,366
U.S. Pat. No. 5,871,986
U.S. Pat. No. 5,916,776
U.S. Pat. No. 5,922,574
U.S. Pat. No. 5,925,565
U.S. Pat. No. 5,925,565
U.S. Pat. No. 5,928,905
U.S. Pat. No. 5,928,906
U.S. Pat. No. 5,932,451
U.S. Pat. No. 5,935,819
U.S. Pat. No. 5,935,825
U.S. Pat. No. 5,939,291
U.S. Pat. No. 5,942,391
U.S. Pat. No. 5,945,100
U.S. Pat. No. 5,958,895
U.S. Pat. No. 5,981,274
U.S. Pat. No. 5,994,624
U.S. Pat. No. 6,008,341
U.S. Pat. No. 6,288,214
U.S. Pat. No. 6,294,177
U.S. Pat. No. 6,651,655
U.S. Pat. No. 6,656,462
U.S. Pat. No. 6,733,754
U.S. Pat. No. 6,756,361
U.S. Pat. No. 6,770,278
U.S. Pat. No. 6,793,923
U.S. Pat. No. 6,814,971
U.S. Pat. No. 6,936,258
U.S. Patent Appln. 2002/0169288
U.S. Patent Appln. 2003/0153022
Studier et al., Methods Enzymol. 185, 60-89, 1990.
Bae et al., Proc. Natl. Acad. Sci. USA 101, 12312-12317, 2004.
Kim et al., J Exp Med 207, 1863-70, 2010.
Cheng et al., PLoS Pathog 6, 8, 2010.
Bae & Schneewind, Plasmid 55, 58-63, 2005
Stranger-Jones et al., Proc. Nat. Acad. Sci. USA 103, 16942-16947, 2006.
Abdallah et al., Mol. Microbiol., 62, 667-679, 2006.
Abdallah et al., Nat. Rev. Microbiol., 5, 883-891, 2007.
Albus et al., Infect. Immun., 59:1008-1014, 1991.
An, J. Virol., 71(3):2292-302, 1997.
Anavi, Sc. thesis from the department of Molecular Microbiology and Biotechnology of the Tel-Aviv University, Israel, 1998.
Andersen et al., J. Immunol., 154, 3359-3372, 1995.
Angel et al., Cell, 49:729, 1987b.
Angel et al., Mol. Cell. Biol., 7:2256, 1987a.
Archer, Clin. Infect. Dis., 26, 1179-1181, 1998.
Atchison and Perry, Cell, 46:253, 1986.
Atchison and Perry, Cell, 48:121, 1987.
Ausubel et al., In: Current Protocols in Molecular Biology, John, Wiley & Sons, Inc, New York, 1996.
Baba et al., J. Bacteriol. 190:300-310, 2007.
Bae and Schneewind, Plasmid, 55:58-63, 2006.
Bae et al., Proc. Natl. Acad. Sci. USA, 101, 12312-12317, 2004.
Banerji et al., Cell, 27(2 Pt 1):299-308, 1981.
Banerji et al., Cell, 33(3):729-740, 1983.
Barany and Merrifield, In: The Peptides, Gross and Meienhofer (Eds.), Academic Press, NY, 1-284, 1979.
Behring E A. Über das Zustandekommen der Diphtherie—Immunität bei Thieren. Deutsche Medzinische Wochenschrift, 16:1145-8, 1890.
Bellus, J. Macromol. Sci. Pure Appl. Chem., A31(1): 1355-1376, 1994.
Berkhout et al., Cell, 59:273-282, 1989.
Birch-Hirschfeld, L. 1934. Über die Agglutination von Staphylokokken durch Bestandteile des Säugetierblutplasmas. Klinische Woschenschrift 13:331.
Bjerketorp et al., FEMS Microbiol. Lett., 234:309-314, 2004.
Blanar et al., EMBO J., 8:1139, 1989.
Bodine and Ley, EMBO J., 6:2997, 1987.
Borrebaeck, In: Antibody Engineering—A Practical Guide, W. H. Freeman and Co., 1992.
Boshart et al., Cell, 41:521, 1985.
Bosze et al., EMBO J., 5(7):1615-1623, 1986.
Boucher and Corey. Clin. Infect. Dis. 46:S334-S349, 2008.
Braddock et al., Cell, 58:269, 1989.
Brown et al., Biochemistry, 37:4397-4406, 1998.
Bubeck Wardenburg and Schneewind. J. Exp. Med. 205:287-294, 2008.
Bubeck-Wardenburg et al., Infect. Immun. 74:1040-1044, 2007.
Bubeck-Wardenburg et al., Proc. Natl. Acad. Sci. USA, 103:13831-13836, 2006.
Bulla and Siddiqui, J. Virol., 62:1437, 1986.
Burke et al., J. Inf. Dis., 170:1110-1119, 1994.
Burlak et al., Cell Microbiol., 9:1172-1190, 2007.
Burts and Missiakas, Mol. Microbiol., 69:736-46, 2008.
Burts et al., Proc. Natl. Acad. Sci. USA, 102:1169-1174, 2005.
Campbell and Villarreal, Mol. Cell. Biol., 8:1993, 1988.
Campere and Tilghman, Genes and Dev., 3:537, 1989.
Campo et al., Nature, 303:77, 1983.
Carbonelli et al., FEMS Microbiol. Lett., 177(1):75-82, 1999.
Cedergren et al., Protein Eng., 6:441-448, 1993.
Celander and Haseltine, J. Virology, 61:269, 1987.
Celander et al., J. Virology, 62:1314, 1988.
Cespedes et al., J. Infect. Dis., 191(3):444-52, 2005.
Champion et al., Science, 313:1632-1636, 2006.
Chandler et al., Cell, 33:489, 1983.
Chandler et al., Proc. Natl. Acad. Sci. USA, 94(8):3596-601, 1997.
Chang et al., Lancet., 362(9381):362-369, 2003.
Chang et al., Mol. Cell. Biol., 9:2153, 1989.
Chatterjee et al., Proc. Natl. Acad. Sci. USA, 86:9114, 1989.
Chen and Okayama, Mol. Cell. Biol., 7(8):2745-2752, 1987.
Cheng et al., FASEB J., 23:1-12, 2009.
Choi et al., Cell, 53:519, 1988.
Cocea, Biotechniques, 23(5):814-816, 1997.
Cohen et al., J. Cell. Physiol., 5:75, 1987.
Cosgrove et al., Infect. Control Hosp. Epidemiol. 26:166-174, 2005.
Costa et al., Mol. Cell. Biol., 8:81, 1988.
Cripe et al., EMBO J., 6:3745, 1987.
Culotta and Hamer, Mol. Cell. Biol., 9:1376, 1989.
Dalbey and Wickner, J. Biol. Chem., 260:15925-15931, 1985.
Dandolo et al., J. Virology, 47:55-64, 1983.
De Villiers et al., Nature, 312(5991):242-246, 1984.
DeBord et al., Infect. Immun., 74:4910-4914, 2006.
DeDent et al., EMBO J. 27:2656-2668, 2008.
DeDent et al., J. Bacteriol. 189:4473-4484, 2007.
Deisenhofer et al., Hoppe-Seyh Zeitsch. Physiol. Chem. 359:975-985, 1978.
Deisenhofer, Biochemistry 20:2361-2370, 1981.
Deschamps et al., Science, 230:1174-1177, 1985.
Devereux et al., Nucl. Acid Res., 12:387-395, 1984.
Diep et al., J. Infect. Dis., 193:1495-1503, 2006a.
Diep et al., Lancet., 367:731-739, 2006b.
Dinges et al., Clin. Microbiol. Rev., 13:16-34, 2000.
Duthie and Lorenz, J. Gen. Microbiol., 6:95-107, 1952.
Edbrooke et al., Mol. Cell. Biol., 9:1908, 1989.
Edlund et al., Science, 230:912-916, 1985.
Ekstedt and Yotis, Ann. N.Y. Acad. Sci., 80:496-500, 1960.
Emorl and Gaynes, Clin. Microbiol. Rev., 6:428-442, 1993.
EP 0786519
EP 497524
EP 497525
Epitope Mapping Protocols In: Methods in Molecular Biology, Vol. 66, Morris (Ed.), 1996.
Fechheimer, et al., Proc Natl. Acad. Sci. USA, 84:8463-8467, 1987.
Feng and Holland, Nature, 334:6178, 1988.
Field and Smith, J. Comp. Pathol., 55:63, 1945.
Firak and Subramanian, Mol. Cell. Biol., 6:3667, 1986.
Foecking and Hofstetter, Gene, 45(1):101-105, 1986.
Fortune et al., Proc Natl. Acad. Sci. USA, 102:10676-10681, 2005.
Foster, Nat. Rev. Microbiol., 3:948-958, 2005.
Fournier et al., Infect. Immun., 45:87-93, 1984.
Fraley et al., Proc. Natl. Acad. Sci. USA, 76:3348-3352, 1979.
Friedrich et al., Nature, 425:535-539, 2003.
Fujita et al., Cell, 49:357, 1987.
GB Appln. 2 202 328
Gilles et al., Cell, 33:717, 1983.
Gloss et al., EMBO J., 6:3735, 1987.
Godbout et al., Mol. Cell. Biol., 8:1169, 1988.
Gomez et al., EMBO J. 26:701-709, 2007.
Gomez et al., J. Biol. Chem. 281:20190-20196, 2006.
Gomez et al., Nature Med. 10:842-8, 2004.
Goodbourn and Maniatis, Proc. Natl. Acad. Sci. USA, 85:1447, 1988.
Goodbourn et al., Cell, 45:601, 1986.
Goodyear and Silverman, J. Exp. Med., 197:1125-1139, 2003.
Goodyear and Silverman, Proc. Nat. Acad. Sci. USA, 101:11392-11397, 2004.
Gopal, Mol. Cell. Biol., 5:1188-1190, 1985.
Gouda et al., Biochemistry, 31(40):9665-72, 1992.
Gouda et al., Biochemistry, 37:129-36, 1998.
Graham and Van Der Eb, Virology, 52:456-467, 1973.
Graille et al., Proc. Nat. Acad. Sci. USA 97:5399-5404, 2000.
Greene et al., Immunology Today, 10:272, 1989
Grosschedl and Baltimore, Cell, 41:885, 1985.
Guinn et al., Mol. Microbiol., 51:359-370, 2004.
Guss et al., Eur. J. Biochem. 138:413-420, 1984.
Harland and Weintraub, J. Cell Biol., 101(3):1094-1099, 1985.
Harlow et al., Antibodies: A Laboratory Manual, Cold Spring Harbor Laboratory, Cold Spring Harbor, N.Y., Chapter 8, 1988.
Hartleib et al., Blood 96:2149-2156, 2000.
Harvey et al., Proc. Natl. Acad. Sci. USA, 83:1084-1088, 1986.
Haslinger and Karin, Proc. Natl. Acad. Sci. USA, 82:8572, 1985.
Hauber and Cullen, J. Virology, 62:673, 1988.
Hen et al., Nature, 321:249, 1986.
Hensel et al., Lymphokine Res., 8:347, 1989.
Herr and Clarke, Cell, 45:461, 1986.
Hirochika et al., J. Virol., 61:2599, 1987.
Hirsch et al., Mol. Cell. Biol., 10:1959, 1990.
Holbrook et al., Virology, 157:211, 1987.
Horlick and Benfield, Mol. Cell. Biol., 9:2396, 1989.
Hsu et al., Proc. Natl. Acad. Sci. USA, 100:12420-12425, 2003.
Huang et al., Cell, 27:245, 1981.
Hug et al., Mol. Cell. Biol., 8:3065, 1988.
Huston et al., In: Methods in Enzymology, Langone (Ed.), Academic Press, NY, 203:46-88, 1991.
Hwang et al., Mol. Cell. Biol., 10:585, 1990.
Imagawa et al., Cell, 51:251, 1987.
Imbra and Karin, Nature, 323:555, 1986.
Imler et al., Mol. Cell. Biol., 7:2558, 1987.
Imperiale and Nevins, Mol. Cell. Biol., 4:875, 1984.
Innis et al., Proc Natl Acad Sci USA, 85(24):9436-9440, 1988.
Inouye and Inouye, Nucleic Acids Res., 13: 3101-3109, 1985.
Jakobovits et al., Mol. Cell. Biol., 8:2555, 1988.
Jameel and Siddiqui, Mol. Cell. Biol., 6:710, 1986.
Jansson et al., FEMS Immunol. Med. Microbiol. 20:69-78 1998.
Jaynes et al., Mol. Cell. Biol., 8:62, 1988.
Jensen, Acta Path. Microbiol. Scandin. 44:421-428, 1958.
Johnson et al., Methods in Enzymol., 203:88-99, 1991.
Johnson et al., Mol. Cell. Biol., 9:3393, 1989.
Jones, Carb. Research, 340:1097-1106, 2005.
Jonsson et al., Oral Dis., 8(3):130-140, 2002.
Joyce et al., Carbohydrate Research 338:903-922 (2003
Kadesch and Berg, Mol. Cell. Biol., 6:2593, 1986.
Kaeppler et al., Plant Cell Rep., 8:415-418, 1990.
Kaneda et al., Science, 243:375-378, 1989.
Karin et al., Mol. Cell. Biol., 7:606, 1987.
Katinka et al., Cell, 20:393, 1980.
Kato et al, J. Biol. Chem., 266:3361-3364, 1991.
Kawamoto et al., Mol. Cell. Biol., 8:267, 1988.
Kennedy et al., Proc. Natl. Acad. Sci. USA 105:1327-1332, 2008.
Kiledjian et al., Mol. Cell. Biol., 8:145, 1988.
Kinoshita, M., N. Kobayashi, S. Nagashima, M. Ishino, S. Otokozawa, K. Mise, A. Sumi, H. Tsutsumi, N. Uehara, N. Watanabe, and M. Endo. 2008. Diversity of staphylocoagulase and identification of novel variants of staphylocoagulase gene in Staphylococcus aureus. Microbiol. Immunol.s 52:334-348.
Klamut et al., Mol. Cell. Biol., 10:193, 1990.
Klevens et al., Clin. Infect. Dis., 2008; 47:927-30, 2008.
Klevens et al., JAMA, 298:1763-1771, 2007.
Koch et al., Mol. Cell. Biol., 9:303, 1989.
Kohler and Milstein, Nature 256:495-497 (1975
Kriegler and Botchan, In: Eukaryotic Viral Vectors, Gluzman (Ed.), Cold Spring Harbor: Cold Spring Harbor Laboratory, NY, 1982.
Kriegler and Botchan, Mol. Cell. Biol., 3:325, 1983.
Kriegler et al., Cell, 38:483, 1984a.
Kriegler et al., Cell, 53:45, 1988.
Kriegler et al., In: Cancer Cells 2/Oncogenes and Viral Genes, Van de Woude et al. eds, Cold Spring Harbor, Cold Spring Harbor Laboratory, 1984b.
Kroh et al., Proc. Natl. Acad. Sci. USA, 106:7786-7791, 2009.
Kuhl et al., Cell, 50:1057, 1987.
Kuklin et al., Infect. Immun., 74:2215-23, 2006.
Kunz et al., Nucl. Acids Res., 17:1121, 1989.
Kuroda et al., Lancet., 357:1225-1240, 2001.
Kyte and Doolittle, J. Mol. Biol., 157(1):105-132, 1982.
Lagergard et al., Eur. J. Clin. Microbiol. Infect. Dis., 11:341-5, 1992.
Lam et al., J. Bacteriol., 86:87-91, 1963.
Lampe et al., EMBO J. 15:5470-5479, 1996.
Larsen et al., Proc Natl. Acad. Sci. USA., 83:8283, 1986, 1963.
Laspia et al., Cell, 59:283, 1989.
Latimer et al., Mol. Cell. Biol., 10:760, 1990.
Lee et al., Nature, 294:228, 1981.
Lee et al., Nucleic Acids Res., 12:4191-206, 1984.
Lee, Trends Microbiol. 4(4):162-166, 1996.
Levenson et al., Hum. Gene Ther., 9(8):1233-1236, 1998.
Levinson et al., Nature, 295:79, 1982.
Lin et al., Mol. Cell. Biol., 10:850, 1990.
Lowy, New Engl. J. Med., 339:520-532, 1998.
Luria et al., EMBO J., 6:3307, 1987.
Lusky and Botchan, Proc. Natl. Acad. Sci. USA, 83:3609, 1986.
Lusky et al., Mol. Cell. Biol., 3:1108, 1983.
Macejak and Sarnow, Nature, 353:90-94, 1991.
MacGurn et al., Mol. Microbiol., 57:1653-1663, 2005.
Maira-Litran et al., Infect. Immun., 70:4433-4440, 2002.
Maira-Litran et al., Vaccine, 22:872-879, 2004.
Majors and Varmus, Proc. Natl. Acad. Sci. USA, 80:5866, 1983.
Markwardt, Untersuchungen über Hirudin. Naturwissenschaften, 41:537-538, 1955.
Mazmanian et al., Mol. Microbiol. 40, 1049-1057, 2001.
Mazmanian et al., Mol. Microbiol., 40(5):1049-1057, 2001.
Mazmanian et al., Proc. Natl. Acad. Sci. USA, 97:5510-5515, 2000.
Mazmanian et al., Science, 285(5428):760-3, 1999.
McLaughlin et al., PLoS Pathog., 3:e105, 2007.
McNeall et al., Gene, 76:81, 1989.
Mernaugh et al., In: Molecular Methods in Plant Pathology, Singh et al. (Eds.), CRC Press Inc., Boca Raton, Fla., 359-365, 1995.
Merrifield, Science, 232(4748):341-347, 1986.
Miksicek et al., Cell, 46:203, 1986.
Mordacq and Linzer, Genes and Dev., 3:760, 1989.
Moreau et al., Carbohydrate Res., 201:285-297, 1990.
Moreau et al., Nucl. Acids Res., 9:6047, 1981.
Moreillon et al., Infect. Immun., 63:4738-4743, 1995.
Moreillon et al., Infect. Immun., 63:4738-4743, 1995.
Mosmann and Coffman, Ann. Rev. Immunol., 7:145-173, 1989.
Muesing et al., Cell, 48:691, 1987.
Musher et al., Medicine (Baltimore), 73:186-208, 1994.
Navarre and Schneewind, J. Biol. Chem., 274:15847-15856, 1999.
Needleman & Wunsch, J. Mol. Biol., 48:443, 1970.
Ng et al., Nuc. Acids Res., 17:601, 1989.
Nicolau and Sene, Biochim. Biophys. Acta, 721:185-190, 1982.
Nicolau et al., Methods Enzymol., 149:157-176, 1987.
Novick, Mol. Microbiol., 48:1429-1449, 2003.
O'Brien et al., Mol. Microbiol. 44:1033-1044, 2002.
O'Seaghdha et al., FEBS J. 273:4831-4841, 2006.
Omirulleh et al., Plant Mol. Biol., 21(3):415-28, 1993.
Ondek et al., EMBO J., 6:1017, 1987.
Ornitz et al., Mol. Cell. Biol., 7:3466, 1987.
Pallen, Trends Microbiol., 10:209-212, 2002.
Palmiter et al., Nature, 300:611, 1982.
Palmqvist et al., Microbes. Infect., 7:1501-11, 2005.
Panizzi et al., J. Biol. Chem., 281:1179-1187, 2006.
PCT Appln. PCT/US89/01025
PCT Appln. WO 00/02523
PCT Appln. WO 00/12132
PCT Appln. WO 00/12689
PCT Appln. WO 00/15238
PCT Appln. WO 01/34809
PCT Appln. WO 01/60852
PCT Appln. WO 01/98499
PCT Appln. WO 02/059148
PCT Appln. WO 02/094868
PCT Appln. WO 03/53462
PCT Appln. WO 04/43407
PCT Appln. WO 06/032472
PCT Appln. WO 06/032475
PCT Appln. WO 06/032500
PCT Appln. WO 07/113,222
PCT Appln. WO 07/113,223
PCT Appln. WO 94/09699
PCT Appln. WO 95/06128
PCT Appln. WO 95/08348
PCT Appln. WO 98/57994
Pearson & Lipman, Proc. Natl. Acad. Sci. USA, 85:2444, 1988.
Pech et al., Mol. Cell. Biol., 9:396, 1989.
Pelletier and Sonenberg, Nature, 334(6180):320-325, 1988.
Perez-Stable and Constantini, Mol. Cell. Biol., 10:1116, 1990.
Phonimdaeng et al., Mol. Microbiol., 4:393-404, 1990.
Picard and Schaffner, Nature, 307:83, 1984.
Pinkert et al., Genes and Dev., 1:268, 1987.
Ponta et al., Proc. Natl. Acad. Sci. USA, 82:1020, 1985.
Porton et al., Mol. Cell. Biol., 10:1076, 1990.
Potrykus et al., Mol. Gen. Genet., 199(2):169-177, 1985.
Pugsley, Microbiol. Rev., 57:50-108, 1993.
Pym et al., Mol. Microbiol., 46; 709-717, 2002.
Pym et al., Nat. Med., 9:533-539, 2003.
Queen and Baltimore, Cell, 35:741, 1983.
Quinn et al., Mol. Cell. Biol., 9:4713, 1989.
Redondo et al., Science, 247:1225, 1990.
Reisman and Rotter, Mol. Cell. Biol., 9:3571, 1989.
Remington's Pharmaceutical Sciences, 18th Ed. Mack Printing Company, 1289-1329, 1990.
Resendez Jr. et al., Mol. Cell. Biol., 8:4579, 1988.
Ripe et al., Mol. Cell. Biol., 9:2224, 1989.
Rippe, et al., Mol. Cell. Biol., 10:689-695, 1990.
Rittling et al., Nuc. Acids Res., 17:1619, 1989.
Roben et al., J. Immunol. 154:6437-6445, 1995.
Rosen et al., Cell, 41:813, 1988.
Sakai et al., Genes and Dev., 2:1144, 1988.
Salid-Salim et al., Infect. Control Hosp. Epidemiol. 24:451-455, 2003.
Sambrook et al., In: Molecular cloning, Cold Spring Harbor Laboratory Press, Cold Spring Harbor, N.Y., 2001.
Schaffner et al., J. Mol. Biol., 201:81, 1988.
Schneewind et al., Cell 70:267-281, 1992.
Schneewind et al., EMBO, 12:4803-4811, 1993.
Schneewind et al., Science, 268:103-6, 1995.
Searle et al., Mol. Cell. Biol., 5:1480, 1985.
Sharp and Marciniak, Cell, 59:229, 1989.
Shaul and Ben-Levy, EMBO J., 6:1913, 1987.
Shaw et al., Microbiology, 150:217-228, 2004.
Sheagren, N. Engl. J. Med. 310:1368-1373, 1984.
Sherman et al., Mol. Cell. Biol., 9:50, 1989.
Shopsin et al., J. Clin. Microbiol., 37:3556-63, 1999.
Sibbald et al., Microbiol. Mol. Biol. Rev., 70:755-788, 2006.
Silverman and Goodyear. Nat. Rev. Immunol., 6:465-75, 2006.
Sjodahl, Eur. J. Biochem. 73:343-351, 1977.
Sjoquist et al., Eur. J. Biochem. 30:190-194, 1972.
Sleigh and Lockett, J. EMBO, 4:3831, 1985.
Smith & Waterman, Adv. Appl. Math., 2:482, 1981.
Smith et al., Brit. J. Exp. Pathol., 28:57, 1947.
Sorensen et al., Infect. Immun., 63:1710-1717, 1995.
Spalholz et al., Cell, 42:183, 1985.
Spandau and Lee, J. Virology, 62:427, 1988.
Spandidos and Wilkie, EMBO J., 2:1193, 1983.
Stanley et al., Proc. Natl. Acad. Sci. USA, 100:13001-13006, 2003.
Stephens and Hentschel, Biochem. J., 248:1, 1987.
Stewart and Young, In: Solid Phase Peptide Synthesis, 2d. ed., Pierce Chemical Co., 1984.
Stranger-Jones et al., Proc. Nat. Acad. Sci. USA, 103:16942-16947, 2006.
Stuart et al., Nature, 317:828, 1985.
Studier et al., Methods Enzymol. 185:60-89 1990.
Sullivan and Peterlin, Mol. Cell. Biol., 7:3315, 1987.
Swartzendruber and Lehman, J. Cell. Physiology, 85:179, 1975.
Takebe et al., Mol. Cell. Biol., 8:466, 1988.
Tam et al., J. Am. Chem. Soc., 105:6442, 1983.
Tavernier et al., Nature, 301:634, 1983.
Taylor and Kingston, Mol. Cell. Biol., 10:165, 1990a.
Taylor and Kingston, Mol. Cell. Biol., 10:176, 1990b.
Taylor et al., J. Biol. Chem., 264:15160, 1989.
Thiesen et al., J. Virology, 62:614, 1988.
Thomson et al., J. Immunol., 157(2):822-826, 1996.
Tigges et al., J. Immunol., 156(10):3901-3910, 1996.
Tigges et al., J. Immunol., 156(10):3901-3910, 1996.
Ton-That et al., Proc. Natl. Acad. Sci. USA, 96(22):12424-9, 1999.
Treisman, Cell, 42:889, 1985.
Tronche et al., Mol. Biol. Med., 7:173, 1990.
Trudel and Constantini, Genes and Dev., 6:954, 1987.
Tyndell et al., Nuc. Acids. Res., 9:6231, 1981.
Uhlen et al., J. Biol. Chem. 259:1695-1702 and 13628 (Corr.) 1984.
van den Ent and Lowe, FEBS Lett., 579:3837-3841, 2005.
van Wely et al., FEMS Microbiol. Rev., 25:437-454, 2001.
Vannice and Levinson, J. Virology, 62:1305, 1988.
Vasseur et al., Proc Natl. Acad. Sci. USA, 77:1068, 1980.
Vaughan, et al., Nat. Biotech. 16; 535-539, 1998.
Wang and Calame, Cell, 47:241, 1986.
Weber et al., Cell, 36:983, 1984.
Weinberger et al. Mol. Cell. Biol., 8:988, 1984.
Weiss et al., J. Antimicrob. Chemother., 53(3):480-6, 2004.
Winoto and Baltimore, Cell, 59:649, 1989.
Wong et al., Gene, 10:87-94, 1980.
Xu et al., J. Infect. Dis., 189:2323-2333, 2004.
Xu et al., Mol. Microbiol., 66(3):787-800, 2007.
Yutzey et al. Mol. Cell. Biol., 9:1397, 1989.

1. A pharmaceutical composition comprising a live, isolated staphylococcal bacteria that does not express a functional SpA, srtA, adsA and/or agrA polypeptide.

2. The pharmaceutical composition comprising a live, isolated staphylococcal bacteria of claim 1, wherein the bacterium does not express adsA.

3. The pharmaceutical composition comprising a live, isolated staphylococcal bacteria of claim 1, wherein the bacterium does not express srtA.

4. The pharmaceutical composition comprising a live, isolated staphylococcal bacteria of claim 1, wherein the bacterium does not express agrA.

5. The pharmaceutical composition comprising a live, isolated staphylococcal bacteria of claim 1, wherein the bacterium does not express SpA and adsA, or SpA and srtA, or agrA and adsA, or SpA and agrA, or srtA and agrA, or adsA and srtA, or SpA, srtA, and adsA, or srtA, adsA and agrA, or SpA, srtA and agrA, or SpA, agrA and adsA, or SpA, srtA, adsA and agrA.

6. The isolated staphylococcal bacterium of claim 1, wherein the bacterium lacks at least part of a coding region for a srtA or agrA polypeptide.

7.-8. (canceled)

9. The isolated staphylococcal bacterium of claim 1, wherein the bacterium further comprises a heterologous drug susceptibility determinant.

10. The isolated staphylococcal bacterium of claim 1, wherein the staphylococcal bacterium is S. aureus.

11.-20. (canceled)

21. A method of making a vaccine comprising the step of formulating a bacterium of the pharmaceutical composition of claim 1.

22.-25. (canceled)

26. A pharmaceutical composition comprising a bacterium according to claim 1.

27. A nucleic acid encoding the genome of a staphylococcal bacterium that does not express a functional SpA, srtA, adsA and/or agrA polypeptide.

28.-35. (canceled)

36. A method for eliciting an immune response against a staphylococcus bacterium in a subject comprising providing to the subject an effective amount of the pharmaceutical composition of claim 1.

37. The method of claim 36, further comprising administering to the subject an adjuvant, cytokine or interleukin biological response modifier.

38.-40. (canceled)

41. The method of claim 36, wherein the vaccine is administered orally, parenterally, subcutaneously, intramuscularly, or intravenously.

42.-45. (canceled)

46. A method for treating a staphylococcal infection in a subject comprising providing to a subject having, suspected of having or at risk of developing a staphylococcal infection an effective amount of a pharmaceutical composition according to claim 1.

47.-48. (canceled)

49. The method of claim 47, wherein the staphylococcal infection is resistant to one or more treatments.

50. The method of claim 49, wherein the staphylococcal infection is a methicillin resistant staphylococcal infection.

51. The method of claim 46, further comprising administering adjuvant, cytokine or interleukin biological response modifier.

52.-54. (canceled)

55. The method of claim 46, wherein the vaccine is administered orally, parenterally, subcutaneously, intramuscularly, or intravenously.

56.-60. (canceled)


SEQUENCE LISTING (2 sequences)

SEQ ID NO: 1 - protein, 206 aa, Staphylococcus aureus
MKKWTNRLMTIAGVVLILVAAYLFAKPHIDNYLHDKDKDEKIEQYDKNVKEQASKDKKQQAKPQIPKDKSKVAGYIEIPDADIKEPVYPGPATPEQLNRGVSFAEENESLDDQNISIAGHTFIDRPNYQFTNLKAAKKGSMVYFKVGNETRKYKMTSIRDVKPTDVGVLDEQKGKDKQLTLITCDDYNEKTGVWEKRKIFVATEVK

SEQ ID NO: 2 - protein, 238 aa, Staphylococcus aureus
MKIFICEDDPKQRENMVTIIKNYIMIEEKPMEIALATDNPYEVLEQAKNMNDIGCYFLDIQLSTDINGIKLGSEIRKHDPVGNIIFVTSHSELTYLTFVYKVAAMDFIFKDDPAELRTRIIDCLETAHTRLQLLSKDNSVETIELKRGSNSVYVQYDDIMFFESSTKSHRLIAHLDNRQIEFYGNLKELSQLDDRFFRCHNSFVVNRHNIESIDSKERIVYFKNKEHCYASVRNVKKI